(19) United States
(12) Patent Application Publication
(10) Pub. No.: US 2016/0235980 A1
(43) Pub. Date: Aug. 18, 2016

(54) SYSTEMS AND METHODS FOR DIRECTED ENERGY THERAPEUTICS

(71) Applicant: NooThera Technologies LLC, Estero, FL (US)

(72) Inventors: Marvin Berman, Plymouth Meeting, PA (US); Michael R. Manning, Langhorne, PA (US); Michael L. Weiner, Estero, FL (US); Warren Woodford, Austin, TX (US)

(21) Appl. No.: 15/018,723

(22) Filed: Feb. 8, 2016

Related U.S. Application Data
(60) Provisional application No. 62/112,838, filed on Feb. 6, 2015.
(60) Provisional application No. 62/112,892, filed on Feb. 6, 2015.
(60) Provisional application No. 62/274,067, filed on Dec. 31, 2015.

(51) Int. Cl. A61N 1/36; A61N 7/00; A61N 5/06; A61N 2/00; A61N 2/02

(52) U.S. Cl. CPC A61N 1/36014; A61N 2/002; A61N 2/006; A61N 1/36025; A61N 2/02; A61N 5/0622; A61N 7/00; A61N 2005/0626; A61N 2005/0659; A61N 2005/0662; A61N 2005/0651; A61N 2005/067

(57) ABSTRACT

The present disclosure relates generally to methods, apparatus and systems for beneficial administration of directed energy to target locations on subject. In some implementations, the directed energy is administered at or near the surface of the body and includes one or more forms of energy including but not limited to visible light, infrared light (IR), near infrared light (NIR), electromagnetic fields (EMF, whether pulsed, repetitive, or static), millimeter wave (MW), ultrasound (US) or other therapeutic wavelengths and/or energy forms.

### CROSS-REFERENCE TO RELATED APPLICATION

This application claims priority to and the full benefit of U.S. Provisional Patent Application Ser. No. 62/112,838, filed Feb. 6, 2015, and titled “DEVICE AND PROCESS FOR TREATING DEMENTIA, INCLUDING ALZHEIMER'S, AND OTHER COGNITIVE DISORDERS”, U.S. Provisional Patent Application Ser. No. 62/112,892, filed Feb. 6, 2015, and titled “DEVICE AND PROCESS FOR TREATING DEMENTIA, INCLUDING ALZHEIMERS, AND OTHER COGNITIVE DISORDERS”, and U.S. Provisional Patent Application Ser. No. 62/274,067, filed Dec. 31, 2015 and titled “METHOD AND APPARATUS FOR INDUCING BIOLOGICAL RESPONSE VIA APPLICATION OF ELECTROMAGNETIC RADIATION” the entire contents of which are incorporated herein by reference.

### FIELD OF THE DISCLOSURE

The present disclosure relates to the non-invasive inducement of biological responses by administration of directed energy therapy. More specifically, the present disclosure describes focused administration of a directed energy to targeted areas of the body, for specific periods of time in order to achieve a desired biological response.

### BACKGROUND OF THE DISCLOSURE

The state of the art in medicine and the treatment human maladies can fairly be described as heavily influenced by and reliant upon pharmacological and surgical means. For certain conditions, it is more heavily weighted towards pharmacological means, such as for cognitive or neurological degenerative conditions as well as pain management. For other conditions, it is more heavily weighted towards surgical means. The drawbacks of pharmacology includes the high cost of drug development, the complexity of managing drug interactions for patients with co-morbidities and multiple medications, and the potential for over-prescription which can lead (and has led) to widespread addiction for painkillers and the associated social, professional and family problems. For surgery, the drawbacks involves lack of productivity with time away from work for pre-operative visits, surgery and post-operative recovery, as well as the resulting pain and potential for infection based on the incision and wound-healing process, etc. . . . . Given these drawbacks, along with the demographics of an increasing population of advanced years and longer life expectancies, it would be beneficial to provide alternate techniques for minimizing our reliance on pharmacological and surgical means.

### SUMMARY OF THE DISCLOSURE

The present disclosure relates generally to methods, apparatus and systems for beneficial administration of directed energy to target locations on subject. In some implementations, the directed energy is administered to the surface of the body and includes one or more forms of energy including but not limited to visible light, infrared light (IR), near infrared light (NIR), electromagnetic fields (EMF, whether pulsed, repetitive, or static), millimeter wave (MW), ultrasound (US) or other therapeutic wavelengths and/or energy forms. As will be described in greater detail below, the application of directed energy therapy to mammalian subjects according to the present invention has a great many potential uses and benefits. Among them include the ability to increase the production of vascular endothelial growth factor (VEGF) to impact angiogenesis and neurogenesis. Another is the ability to selectively regulate the generation of adenosine triphosphate (ATP) in the mitochondria of the cells subject to the directed energy therapy. Another is the ability to selectively regulate the efficacy and/or potency of pharmaceuticals, over-the-counter supplements, and/or medications to be administered to or by the subject. A still further is the ability to selectively regulate or induce the generation of stem cells systemically and/or at a particular region of interest within the subject. Yet another is the ability to regulate natural DNA sequences, synthetic DNA sequences, and associated genes, sub-sets and/or pre-cursors of natural and/or synthetic DNA sequences. Based on these wide-ranging abilities, the directed energy therapy of the present disclosure can effect a desired biological response in any of a variety of medical conditions including but not limited to cognitive conditions (e.g. Alzheimer's, dementia, Parkinson's, traumatic brain injury (TBI), chronic traumatic encephalopathy (CTE), post-traumatic stress disorder (PTSD), tinnitus, etc. . . . ), pain conditions (e.g. joint pain, back pain, fibromyalgia, etc. . . . ), inflammatory conditions (e.g. arthritis, etc. . . . ) and any others that would benefit from the various forms of biological regulation set forth above.

According to one aspect of the disclosure, an apparatus for providing directed energy therapy to a mammalian subject comprises a directed energy emission device, a source of directed energy, a controller, a biometric measurement device, and a source of electrical logic signals in logical communication with the controller. The directed energy emission device is positionable proximate to a surface of a region of interest of the subject, and includes a plurality of energy-emitting portals. The source of directed energy is in communication with the plurality of energy-emitting portals and is capable of emitting energy in the form of visible light, infrared light, near infrared light, electromagnetic fields, ultrasound, and/or millimeter waves directed towards the region of interest via the energy-emitting portals of the directed energy emission device. The controller is in logical communication with the source of directed energy and is operative to cause the source of directed energy to provide a predetermined directed energy therapy to at least some of the energy-emitting portals, wherein the predetermined directed energy therapy comprises delivering at least one type of directed energy from a specific selection of energy-emitting portals for a predetermined period of time with a predetermined energy level. The controller further includes a processor and digital data storage.

The biometric measurement device is capable of quantifying one or more biological aspects of the subject, and is in logical communication with the controller (either constantly or intermittently) to provide digital data to the controller indicative of the one or more measurements quantifying biological aspects of the subject before, during, and/or after the administration of the predetermined directed energy therapy. The digital data may be used by a neurofeedback system, a biofeedback system, and/or a data storage device. The neurofeedback system is in logical communication with the controller and includes external stimulation administered to the subject. Non-limiting example forms of external stimulation include visual stimulation, audio stimulation, trans-cranial electrical stimulation, trans-cranial magnetic stimulation, trans-tongue electrical stimulation, and/or trans-dermal electrical stimulation. The biofeedback system is in logical communication with the source of directed energy to either continue unchanged or modify the predetermined directed energy therapy and/or neurofeedback administered to the subject based on the one or more measurements quantifying biological aspects of the subject. The data storage device is used for storing the one or more measurements quantifying biological aspects from the subject before, during, and/or after the predetermined directed energy therapy and/or neurofeedback therapy is administered to the subject.

The source of electrical logic signals is in logical communication with the controller and is capable of causing the controller to receive input from the biometric measurement device and cause the source of directed energy to continue delivering the predetermined directed energy therapy or selectively modify the predetermined directed energy therapy to the subject based on the input from the biometric measurement device. Modifying the predetermined directed energy therapy includes modifying the selection of the energy portals, modifying the type of directed energy, modifying the level of directed energy, modifying the duration of the directed energy, and/or modifying the frequency of the directed energy.

According to another aspect, the controller is additionally in logical communication with a digital communications network and the source of electrical logic signals includes at least one network access device communicating the electrical logic signals via the digital communications network to deliver the predetermined directed energy therapy to the subject at a location remote from the controller.

According to another aspect, the network access device additionally receives and delivers the digital data indicative of the feedback from the biometric measurement device to the controller at a location remote from the subject.

According to another aspect, the energy emission portals comprise one or more of: (a) a plurality of light sources capable of emitting light with a wavelength of between 450 nanometers and 1500 nanometers, wherein the light sources include at least one of light emitting diodes and laser diodes; (b) a plurality of fiber optic transmission elements each having a distal end for providing luminous communication from a source of light with a wavelength of between 450 nanometers and 1500 nanometers to the respective energy emission portal, wherein the source of light includes at least one of a light emitting diode, a laser diode, a fixed frequency laser, and a variable frequency laser; (c) one or more ultrasound transducers; (d) one or more millimeter wave transducers; (e) one or more electrodes for delivering at least one of pulsed direct current (DC) and alternating current (AC); (f) one or more electromagnetic coils capable of emitting at least one of pulsed and static electromagnetic fields; and (g) combinational transducers capable of delivering any combination of types of directed energy.

According to another aspect, the multiple energy emitting portals comprise a matrix in which at least some of the matrix of energy emitting portals may be activated by the controller to emit directed energy independent of other energy emitting portals.

According to another aspect, the multiple energy emitting portals comprising the matrix are identifiable via predetermined coordinates selected from one or more coordinate types including Cartesian, Polar, number line, cylindrical, spherical, homogenous, curvilinear, orthogonal, skew log-polar, Plucker, canonical, parallel, barycentric, trilinear, and any transformation thereof, and the controller may activate one or more of the energy emitting portals according to a pattern that may be associated with multiple predetermined coordinates.

According to another aspect, the controller activates the source of directed energy to temperospatially deliver directed energy via emitting portals positioned proximate to a first portion of the region of interest and/or a second portion of the region of interest, and the biometric measurement device provides feedback indicative of stimulation of and/or reduced activity in one or both of the first and second portions of the region of interest and adjusts the selection of energy emitting portals, the type of directed energy, the duration of the directed energy, and/or the level of the directed energy based on the feedback.

According to another aspect, the directed energy emission device forms part of a multi-modal wearable cranial appliance, a wearable orthopedic appliance, a sleep-related appliance, an article of clothing, a patient imaging system, a hand-held device, a portable controller for administration and monitoring by a remote practitioner, and/or a wireless controller system.

According to another aspect, the biometric measurement device comprises an electronic gaming device requiring inputs based upon cognitive analysis and manipulation of a user input mechanism.

According to another aspect, the biometric measurement device comprises an electroencephalography (EEG) apparatus, an electromyography (EMG) apparatus, a thermometer, an electrodermography (EDG) apparatus, a photoplethysmography (PPG) apparatus, an electrocardiogram (ECG) apparatus, a pneumography apparatus, a capnography apparatus, a rheonocephalography (REG) apparatus, and/or a hemoencephalography (HEG) apparatus.

According to another aspect, the predetermined directed energy therapy is capable of regulating the production of adenosine triphosphate (ATP) and is selected to up regulate and/or down regulate the production of ATP of cells within the target region of the subject.

According to another aspect, the predetermined directed energy to up regulate ATP production is selected having a peak wavelength of approximately 620 nanometers, approximately 680 nanometers, approximately 760 nanometers, and/or approximately 820 nanometers.

According to another aspect, the predetermined directed energy to down regulate ATP production is selected having a peak wavelength of approximately 750 nanometers, approximately 870 nanometers, approximately 900 nanometers, and/or approximately 950 nanometers.

According to another aspect, the predetermined directed energy therapy is capable of regulating the efficacy and/or potency of pharmaceuticals, over-the-counter supplements, and/or medications to be administered to or by the subject and is selected to up regulate and/or down regulate the efficacy and/or potency of pharmaceuticals, over-the-counter supplements, and/or medications to be administered to or by the subject.

According to another aspect, the predetermined directed energy therapy is capable of regulating endogenous stem cells, natural DNA sequences, synthetic DNA sequences, and/or associated genes, sub-sets and/or pre-cursors of the natural or synthetic DNA sequences and is selected to up regulate and/or down regulate endogenous stem cells, natural DNA sequences, synthetic DNA sequences, and/or associated genes, sub-sets and/or pre-cursors of said natural or synthetic DNA sequences.

According to another aspect, the predetermined directed energy therapy is selected: (a) from a pre-existing list of potential directed energy therapies based on a pre-therapy assessment of the subject with reference to an aggregated database incorporating pre-therapy assessments of other subjects, directed energy therapies of other subjects, and/or results of the directed energy therapies of other subjects; and/or (b) by a user of the apparatus based on a pre-therapy assessment of the subject with and/or without reference to the aggregated database.

According to another aspect, the pre-therapy assessment includes positron emission tomography (PET), computed tomography (CT), single-photo emission computed tomography (SPECT), blood biomarker testing, and/or cognitive testing to establish a functional baseline for the subject from which to compare the results of the predetermined directed energy therapy as determined by the biometric measurement device during and/or after the administration of the predetermined directed energy therapy.

According to another aspect, the effectiveness of the directed energy therapy may be augmented by physical exercise and/or mental exercise before, during and/or after the directed energy therapy.

According to another aspect, the directed energy emission device, the source of directed energy, the controller and the biometric measurement device are all part of a clinical directed energy system located in a first location, and a remote directed energy system is located at a second location remote from the first location. The remote directed energy system includes a second directed energy emission device, a second source of directed energy, a second controller, and a second biometric measurement device to collectively deliver the predetermined directed energy therapy as originated by the controller of the clinical directed energy system to the subject using the remote directed energy system.

According to another aspect, the predetermined directed energy therapy is capable of regulating the efficacy and/or potency of pharmaceuticals, over-the-counter supplements, and/or medications to be administered to or by the subject and is selected to up regulate and/or down regulate the efficacy and/or potency of pharmaceuticals, over-the-counter supplements, and/or medications to be administered to or by the subject. The clinical directed energy system is capable of: (a) accessing electronic health records of the subject before the application of the predetermined directed energy therapy; (b) calculating the characteristics of the energy of the predetermined directed energy therapy to be administered to the subject, wherein the characteristics of the energy include at least one of fluence, flux density, wavelength, waveform, pulse rate, and duration; (c) calculating a degree of modification to the amount of pharmaceutical, over-the-counter supplement and/or medication to be administered to the subject based on the increase or decrease in efficacy and/or potency due to the up or down regulation as a result of the predetermined directed energy therapy; and (d) communicating the degree of modification to a prescribing healthcare professional, a pharmacy associated with the subject, and/or the remote directed energy system.

Still other aspects are described in greater detail below.

### BRIEF DESCRIPTION OF THE DRAWINGS

The accompanying drawings, that are incorporated in and constitute a part of this specification, illustrate several embodiments of the disclosure and, together with the description, serve to explain the principles of the disclosure:

FIG. 1 illustrates a flow chart of a method for providing therapeutic administration of directed energy to a subject in accordance with some embodiments.

FIG. 2 is a block diagram illustrating a system for providing therapeutic administration of directed energy to a subject in accordance with some embodiments.

FIG. 3 illustrates a block diagram of a controller configured for providing therapeutic administration of directed energy to a subject in accordance with some embodiments.

FIG. 4 illustrates a device for providing therapeutic administration of directed energy to a cranium of a subject in accordance with some embodiments.

FIG. 5 is a block diagram illustrating a system for providing therapeutic administration of directed energy to a subject via a plurality of additional options.

FIGS. 6-10 illustrate a variety of different orthopedic appliances for use in providing therapeutic administration of directed energy in accordance with certain embodiments.

FIGS. 11-14 illustrate a variety of rest-related appliances (sheet, pillows) for use in providing therapeutic administration of directed energy in accordance with some embodiments.

FIGS. 15-16 illustrate a variety of articles of clothing for use in providing therapeutic administration of directed energy in accordance with certain embodiments.

FIGS. 17-18 illustrate a variety of multiple resonance therapy (MRT) systems for use in providing therapeutic administration of directed energy in accordance with certain embodiments.

FIG. 19 illustrates a hand-held system for use in providing therapeutic administration of directed energy in accordance with certain embodiments.

FIG. 20 illustrates an implantable device for use in providing therapeutic administration of directed energy in accordance with certain embodiments.

FIG. 21 illustrates a portable directed energy system for use in providing therapeutic administration of directed energy in accordance with certain embodiments.

FIG. 22 illustrates a telemedicine system for providing therapeutic administration of directed energy in accordance with certain embodiments.

FIGS. 23-24 illustrates a wireless control system for providing therapeutic administration of directed energy to a subject in accordance with some embodiments.

### DETAILED DESCRIPTION

The present disclosure relates generally to methods, apparatus and systems for beneficial administration of directed energy to target locations on subject. In some implementations, the directed energy is administered to the surface of the body and includes one or more forms of energy including but not limited to visible light, infrared light (IR), near infrared light (NIR), electromagnetic fields (EMF, whether pulsed, repetitive, or static), millimeter wave (MW), ultrasound (US) or other therapeutic wavelengths and/or energy forms. As will be described in greater detail below, the application of directed energy therapy to mammalian subjects according to the present invention has a great many potential uses and benefits. Among them include the ability to increase the production of vascular endothelial growth factor (VEGF) to impact angiogenesis and neurogenesis. Another is the ability to selectively regulate the generation of adenosine triphosphate (ATP) in the mitochondria of the cells subject to the directed energy therapy. Another is the ability to selectively regulate the efficacy and/or potency of pharmaceuticals, over-the-counter supplements, and/or medications to be administered to or by the subject. A still further is the ability to selectively regulate or induce the generation of stem cells systemically and/or at a particular region of interest subject to the directed energy therapy (e.g. in the brain or any intervening structures, such as in the red blood of the bone of the skull). Yet another is the ability to regulate natural DNA sequences, synthetic DNA sequences, and associated genes, sub-sets and/or pre-cursors of natural and/or synthetic DNA sequences. Based on these wide-ranging abilities, the directed energy therapy of the present disclosure can effect a desired biological response in any of a variety of medical conditions including but not limited to cognitive conditions (e.g. Alzheimer's, dementia, Parkinson's, traumatic brain injury (TBI), chronic traumatic encephalopathy (CTE), post-traumatic stress disorder (PTSD), tinnitus, etc. . . . ), pain conditions (e.g. joint pain, back pain, fibromyalgia, etc. . . . ), inflammatory conditions (e.g. arthritis, etc. . . . ) and any others that would benefit from the various forms of biological regulation set forth above.

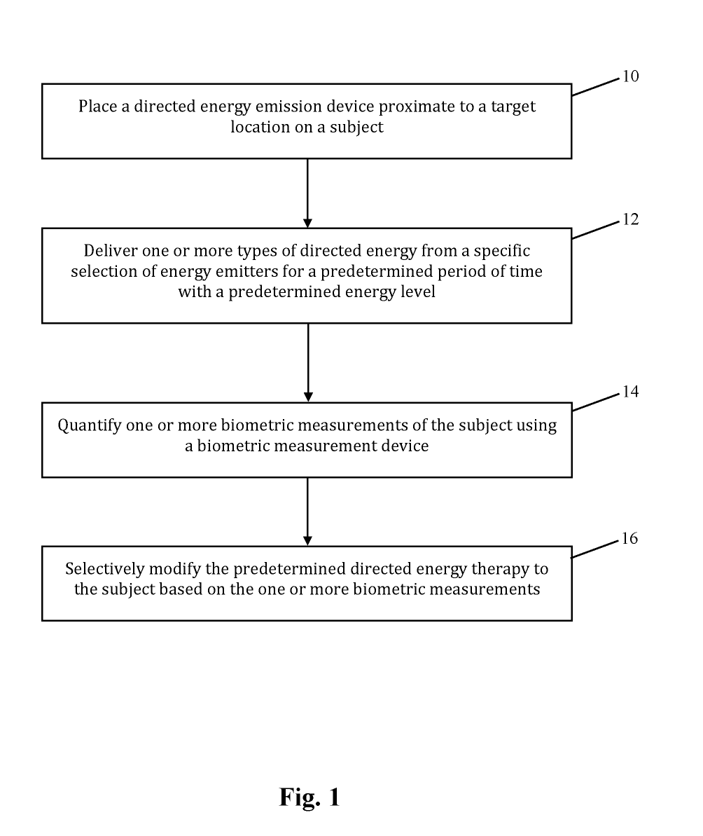

FIG. 1 illustrates a general overview of a therapeutic directed energy treatment protocol. The first step 10 is to place a directed energy emission device proximate to a target location on a subject. The second step 12 is to deliver one or more forms of directed energy from a specific selection of energy emitters for a predetermined period of time with a predetermined energy level. The directed energy then penetrates the surface and underlying tissue, where it can achieve the desired therapeutic impact. The third step 14 is to quantify one or more biometric measurements of the subject using a biometric measurement device. As will be described in greater detail below, a “biometric measurement device” as used herein is meant to include any device, sensor, or system capable of acquiring or quantifying one or more biological aspects of the subject, wherein biological aspects of the subject may include any of a variety of biological parameters or characteristics (e.g. physical, cognitive, behavioral, etc. . . . ). These biometric measurements may be assessed before treatment in order to establish a baseline from which to compare against over time, as well as analyzed in real time or periodically during the administration of directed energy therapy and/or after the treatment session. The biometric measurements may be acquired and/or assessed manually (e.g. by a physician, trained therapist, etc. . . . ) and/or automatically (e.g. via any of a variety of biosensors or biofeedback systems for use within or by a computer program. This leads to the fourth step 16 in which the directed energy therapy program can be continued as originally determined or selectively modified based on the one or more biometric measurements.

In some implementations, a controller provides control signals to a source of directed energy administered to the subject. The controller may also receive instruction from a remote operator, such as a health care practitioner located in a remote location.

In various applications, administration of directed energy may be used in treatment of medical conditions such as, for example, one or more of: Traumatic Brain Injury (TBI), Chronic Traumatic Encephalopathy (CTE), Post Traumatic Stress Disorder (PTSD), depression, tinnitus, and various forms of neurodegenerative diseases (e.g. Alzheimer's, dementia, Parkinson's, etc. . . . ). The use of the methods and apparatus disclosed herein may result in one or more of diminishing, stabilizing and reversal of symptoms of these medical conditions, alone or in combination with chemical intervention and/or neurofeedback techniques for brain entrainment, retraining and/or enhancement. Symptoms such as loss of memory attributable to Alzheimer's disease and problems resulting from brain damage such as traumatic brain injury (TBI) may also be alleviated. Symptoms associated with a variety of other neurocognitive and disruptive conditions, such as, for example, ADD, ADHD and bipolar disorder may also be relieved through application of the innovation described herein. Although much of the current directed energy therapy involves the brain, other conditions may also benefit from the present disclosure including but not limited to osteomyelitis, bone virus, sepsis, bleeding, infection, tissue damage, muscle damage, erectile dysfunction, and cancer.

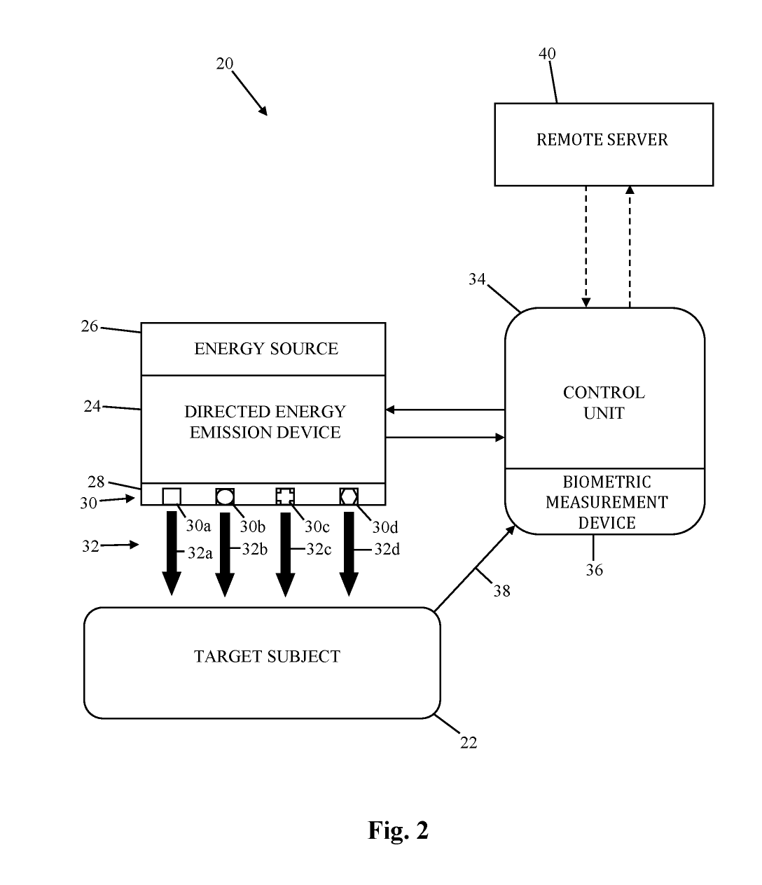

Referring now to FIG. 2, a block diagram illustrates components of a system 20 that is useful for administering directed energy to a specific location on a target subject 22. The directed energy may be administered via a directed energy emission device 24 positioned proximate a target location on the subject 22. By way of example, the directed energy emission device 24 includes an energy source 26 and a directed energy emitter array 28. The directed energy emitter array 28 comprises a plurality of energy emitting portals 30 (also referred to as “emitters 30”) arranged systematically such that the directed energy emission device 24 is capable of directing several forms of directed energy at one or more target sites on the subject 22. Portals (or emitters) 30 may comprise any structure or component capable of delivering directed energy to a target subject. “Directed energy” as used herein means any form of energy (e.g. light, sound, electrical, magnetic, etc. . . . ) that is directed or otherwise caused to be applied to an anatomical aspect or region of interest in a mammalian subject. In some aspects, the portals 30 may be dual purpose, in that they may send and receive signals. Generally, there may be a different kind of portal 30 present for each type of directed energy 32. For example, in a directed energy emission device 24 that has four kinds of portals 30a-30d, portal 30a would be capable of emitting one form of energy 32a, portal 30b would emit a second form of energy 32b, portal 30c would emit a third form of energy 32c, and portal 30d would emit a fourth form of energy 32d. When the directed energy emission device 24 is positioned proximate to the target subject, the energy emitting portals 30 are directed towards the surface of the target areas in a fixed position relative to the surface of the target subject 22. Although shown where the energy portals 30a-30d are positioned at the same distance from the target subject 22, it should be appreciated that the distance between the specific energy portals 30a-30d and the target subject 22 may depend upon a host of factors, including but not limited to the intended tissue or anatomical target of the directed energy, the type of directed energy, and the level or strength of that particular type of directed energy 32a-32d. As a result, the term “positioned proximate to the target subject” may depend upon any number of the aforementioned factors, as will be appreciated by those skilled in the art.

Directed energy may be any suitable form of energy capable of being applied to effect a desired biological response according to the present invention, including but not limited to visible light, infrared light, near infrared light, electrical stimulation (e.g. pulsed DC or AC electrical stimulation), electromagnetic fields (static or pulsed), ultrasound, and millimeter waves. An energy-emitting portal 30 is in communication with a source of directed energy 26. The source of directed energy 26 may include any number of components, circuitry, electrical hardware and/or software, elements and/or power sources capable of generating and delivering the types of directed energy contemplated by the present disclosure to the target subject 22 via an energy-emitting portal 30. For example, the source of directed energy 26 may include one or more electromagnetic coils for converting electrical current into an electromagnetic field having any of a variety of desired characteristics (e.g. wavelength, power, flux, etc. . . . ), one or more sources of light (e.g. lasers, light-emitting diodes (LED, etc. . . . ) for generating light having any of a variety of desired characteristics (e.g. wavelengths in the visible, infrared, and near-infrared range, etc. . . . ), one or more ultrasound transducers for generating sonic energy having any of a variety of desired characteristics (e.g. wavelengths, power, etc. . . . ), and one or more sources of electromagnetic radiation for generating millimeter waves having any of a variety of desired characteristics (wavelength, power, etc. . . . ). Additional features that help shape or direct the energy may also be present. For example, an energy emitting portal 30 may be in communication with a source of directed energy 26 that is an infrared (“IR”) LED. In some embodiments, then, an LED may be mounted in close proximity to a lens or a lenslet that directs the IR radiation produced by the IR LED to a specific area of the target subject 22. Accordingly, in some embodiments, a lens may focus IR radiation produced an LED and in other embodiments, a lens may disperse IR radiation emitted by an IR LED. An energy-emitting portal 30 may also include a distal end of a fiber optic conveying directed energy 32 with specific characteristics, such as IR light of a particular bandwidth.

A controller 34 is placed in logical communication with the energy source 26 and is operative to cause the energy source 26 to provide a predetermined directed-energy therapy to at least some of the energy emitting portals 30, wherein the predetermined directed energy therapy may include delivering one or more types of directed energy 32 from a specific selection of energy portals 30 for a predetermined period of time with a predetermined energy level. In some embodiments, a specific selection of energy portals 30 specifies less than all of the energy emitting portals 30. As described in more detail below, the controller 34 includes a processor and a source of electrical logic, such as a digital storage device. Electrical logic may include a pattern of electrical signals that may be interpreted in a logical manner. In some instances, the energy source 26 may be housed in the controller 34. In other instances, the energy source 26 may be attached to or incorporated within the emission device 24 itself.

A biometric measurement device 36 may be included for acquiring or quantifying one or more biological aspects of the subject 22, which can include physical, cognitive and behavioral aspects (e.g. parameters, characteristics, measurements, etc. . . . ) of the subject. Examples of physical aspects include, but are not necessarily limited to, biological parameters capable of being measured or otherwise assessed via any number of suitable biometric measurement techniques, including but not limited to electroencephalography (EEG, whether standard or quantitative), electromyography (EMG), thermometry, electrodermography (EDG), photoplethysmography (PPG), electrocardiography (ECG), pneumography, capnography, rheonocephalography (REG), and hemoencephalography (HEG). Examples of cognitive aspects include, but are not necessarily limited to, the results of cognitive testing. Examples of behavioral aspects include, but are not necessarily limited to, assessments regarding mood, attentiveness, and physical activity. The biometric measurement device 36 may be a standalone device or incorporated into the controller.

Once acquired, the various biological aspects of the target subject 22 may be used in any of a variety of suitable manners according to an aspect of the present invention. These include, but are not necessarily limited to, using acquired biological aspects of the target subject 22 with a neurofeedback system (not shown) in logical communication with the control unit 34 and which includes any of a variety of suitable types of external stimulation to be administered to the target subject 22 (e.g. visual stimulation, audio stimulation, trans-cranial electrical stimulation, trans-cranial magnetic stimulation, trans-tongue electrical stimulation, and/or trans-dermal electrical stimulation). The combination of directed-energy therapy and neurofeedback may be particularly useful in the treatment of cognitive or other neurological disorders or conditions (e.g. dementia, Alzheimer's, PTSD, TBI, CTE, PTSD, etc. . . . ). To facilitate this, a cranial applicance may be provided that combines the necessary components to achieve both directed-energy therapy and neurofeedback, as well as certain pre-therapy assessments, according to an aspect of the invention. The biological aspects of the target subject 22 may also, again by way of example only and not limitation, be used with a biofeedback system in logical communication with the energy source 26 to continue or modify the predetermined directed energy therapy. A still further example (again, without limitation) involves using the biological aspects with a data storage device for storing such information before, during, and/or after the administration of the predetermined directed energy therapy and/or neurofeedback therapy administered to the subject as described above.

A source of electrical logic signals may be placed in logical communication with the controller 34 which is capable of causing the controller 34 to receive input 38 from the biometric measurement device 36 and cause the source of directed energy 26 to continue delivering the predetermined directed energy therapy via the energy emission device 24 or selectively modify the predetermined directed energy therapy to the subject 22 based upon the input 38 from the biometric measurement device 36. Modifying the predetermined directed energy therapy may include any number of suitable manners, including but not limited to modifying the selection of the energy portals 30a-30d, the type of directed energy 32a-32d, the level (that is, magnitude or amount) of directed energy 32a-32d, the duration of the directed energy 32a-32d, and the frequency of the directed energy 32a-32d.

According to an aspect of the invention, the controller 34 may be in communication with a remote server 40 via a data network such as the Internet. The remote server 40 may be used to store any number of types of data for use with the directed energy therapy system 20. The types of data may include, but are not necessarily limited to, data representing a library of pre-established directed energy therapy options and/or aggregate patient treatment data. If a library of pre-established directed therapy options is employed, a user of the system 20 (e.g. physician, therapist, caregiver, subject, etc. . . . ) may select from any of a number of suitable options depending upon any number of suitable considerations, including but not limited to the desired outcome for the target subject 22 (e.g. improving cognition, reducing or eliminating tinnitus, knee pain, etc. . . . ) and/or a pre-therapy assessment of the target subject 22. If aggregate patient treatment data is employed, such as may be found a normative database based on collected data from a multiplicity of patients, then the aggregate patient data may be used to establish an aggregate baseline that may be compared to the results of pre-therapy, intra-therapy and post-therapy assessments of the target subject 22.

Any number of suitable pre-therapy assessments may be conducted according to an aspect of the invention. These may include, but are not necessarily limited to, elelectroencepholograhy (EEG, whether standard or quantitative/QEEG), positron emission tomography (PET), computed tomography (CT), single-photo emission computed tomography (SPECT), blood biomarker testing, and cognitive testing. Pre-therapy assessments can be used to establish an individual baseline for the subject 22 from which to compare the intra-therapy and/or post-therapy results of subject 22 after having completed the predetermined directed energy therapy. Pre-therapy assessments can also be used to establish an individual baseline for the subject 22 from which to compare against the aggregate baseline established from the aggregate patient data in the remote server 40.

It will be appreciated that, although described above with reference to remote server 40, the control unit 34 may have sufficient local data storage that will allow the same types and uses of the data as described above with reference to remote server 40. This may allow the system to be either operated independently from any remote system or not form part of a remote system, which is also one aspect the present invention.

In some embodiments, the directed energy system 20 may be employed to administer a predetermined directed energy therapy to the cranium of the target subject 22, such as may be desirable to treat any of a variety of cognitive or other neurological disorders or conditions (e.g. dementia, Alzheimer's, PTSD, TBI, CTE, PTSD, etc. . . . ). The directed energy 32 will penetrate into the brain after passing through the scalp and skull of the subject 22. The administration of the directed energy 32 into the brain may induce a host of effects or changes on the neurons and other cells in the brain (e.g. glial cells). For example, the effective penetration of directed energy into the cranium of the subject 22 may be used to selectively regulate (up and/or down depending upon the desired outcome) any number of important chemicals and/or cells in the brain, including inducing the generation of adenosine triphosphate (ATP) in the mitochondria of the cells subject to the directed energy therapy.

For example, directed energy in the form of infrared light may be used to up regulate (increase) ATP production when administered with a peak wavelength of approximately 620 nanometers, approximately 680 nanometers, approximately 760 nanometers and/or 820 nanometers, whereas it may be used to down regulate (decrease) ATP production when administered with a peak wavelength of approximately 750 nanometers, 870 nanometers, 900 nanometers and/or 950 nanometers. Up regulation of ATP may be desired to slow, start or reverse the progression of neurodegenerative conditions such as dementia, Alzheimer's, CTE, etc. . . . . Down regulation of ATP may be beneficial immediately or shortly following acute trauma events to the brain (e.g. TBI, concussions, etc. . . . ) to slow the level of apoptosis of brain cells that may otherwise occur if those cells aren't slowed down based on the down regulation of ATP. This could be used in conjunction with blood biomarkers currently being used to establish whether concussions have occurred. More specifically, if in close time proximity to the trauma it is determined via the administration of a blood biomarker test that a concussion has occurred, it may be beneficial to down regulate ATP in the brain cells in order to minimize the impact of the concussion and allow the brain cells to recover in a controlled manner by the selective up regulation of ATP after the initial down regulation that was effected close in time to the acute trauma event.

Beyond the regulation of ATP, the effective penetration of directed energy into the cranium of the subject 22 may also activate vascular endothelial growth factor (VEGF) to impact angiogenesis and neurogenesis (e.g. neural stem cells), as well as induce the generation of stem cells within the brain and in the red blood cells within the bone of the skull. It may also induce the generation of natural DNA sequences, synthetic DNA sequences, and associated genes, sub-sets and/or pre-cursors of natural and/or synthetic DNA sequences within the brain. Again, the regulation of these compounds and/or cells may be particularly useful in treating any of a variety of cognitive or neurodegenerative conditions, whether a subject is symptomatic or not.

Beyond the benefits for trans-cranial applications, directed energy therapy is also useful in a great many non-cranial locations throughout the body. For example, directed energy therapy may help ailing tissue (e.g. organs) if administered so as to up regulate ATP, stem cells, and trigger DNA/RNA expression to thereby speed the recovery or healing process and/or slow a downward progression. Directed energy stimulation has been shown to induce production of opiates, which can reduce pain and speed up tissue regeneration if accompanied with an up regulation of stem cells (per above). Certain forms and levels of directed energy may also affect the circulatory system by causing vasodilation (which helps improve blood flow) or vasoconstriction (which helps decrease blood flow). Directed energy can also be used to reduce inflammation. Wavelength, power, and direction can be used to target a specific area and activate a desired metabolic process.

Directed energy therapy is also capable of regulating the efficacy and/or potency of pharmaceuticals, over-the-counter supplements, and/or medications to be administered to or by the subject by virtue of the ability to selectively up and/or down regulate the various cellular chemicals, stem cells and aspects of cellular function described above. In one embodiment, the directed energy system 20 may be located in a clinical setting (e.g. physician's office), while another directed energy system (not shown but similar to directed energy system 20) may be located in a remote setting (e.g. home of subject 22) but either remotely monitored or administered or controlled by the clinical system via known or later-developed telemedicine technology or methodology. In this scenario, the clinical directed energy system may be capable of: (a) accessing electronic health records of the subject 22 before the application of the predetermined directed energy therapy; (b) calculating the overall quanta of energy of the predetermined directed energy therapy to be administered to the subject 22; (c) calculating a degree of modification to the amount of pharmaceutical, over-the-counter supplement and/or medication to be administered to the subject 22 based on the increase or decrease in efficacy or potency due to the up or down regulation as a result of the predetermined directed energy therapy; and (d) communicating the degree of modification to at least one of a prescribing healthcare professional, a pharmacy associated with the subject, and the remote directed energy system.

Neurofeedback techniques, direct neural stimulation, and other sensory stimulation interventions can entrain and/or enhance the use and growth of desired neural pathways. Quantitative EEG analysis can be used to identify targets for neuromodulation and subsequent retraining. Other biofeedback techniques may be used to quantitatively analyze treatment effects in areas other than the brain, for example by measuring skin tension, electrical conductivity, breath analysis, muscle tension and integrity, cell surface potential, pH, salt, and other chemical sensitivity. Verbal feedback from that patient can also be used.

In some embodiments, initially, a clinical diagnosis of a subject 22 may be performed in order to identify a treatable malady. The clinical diagnosis may be based on results of tests conducted using diagnostic devices such as, but not limited to, electroencephalography devices (EEG, whether standard or quantitative/QEEG), ultrasound scanners, MRI scanners, SPECT scanners, CT scanners, EEG readers, PET scan and so on. Further, the clinical diagnosis may also be based on behavioral tests conducted based on questionnaires, interviews, cognitive exercises, physical exercises and so on.

Subsequent to identifying the treatable malady, in some embodiments, one or more regions of the brain associated with the functionality affected by the malady may be identified. For example, in case the clinical diagnosis reveals a condition of dementia, one or more of the frontal lobe, the fronto-temporal lobe and the parietal lobe may be identified for treatment.

In some embodiments, a profiling of the subject 22 may be performed in order to identify one or more of physical characteristics, mental characteristics, emotional characteristics, behavioral characteristics, symptom characteristics, historical treatment characteristics and so on. Further, in some embodiments, a profile of the subject 22 generated based on profiling may be used to query a database in order to identify a treatment plan. Accordingly, the database may include records of various treatment plans indexed according to profiles of patients. For instance, in some embodiments, treatment plans may be indexed according to a brain disorder, a stage of the brain disorder, gender of patient, age of patient and so on. Furthermore, in some embodiments, the profile of the subject 22 may be compared with one or more profiles of other patients who received efficacious treatment. Accordingly, based on a match between the profile of the subject 22 with an earlier patient, the treatment plan found effective for the earlier patient may be identified as suitable for the subject 22.

Additionally, in some embodiments, subsequent to retrieving a treatment plan from a database, a medical practitioner may be enabled to modify the treatment plan in order to tailor it for the subject 22. Alternatively, in some embodiments, based on the clinical diagnosis and/or the profile of the subject 22, a medical practitioner may devise the treatment plan specific to the subject 22. However arrived at (e.g. selected, devised, etc. . . . ), once the treatment plan is decided upon as being the one to administer to the subject 22 (e.g. selected or devised) it may thereafter be referred to as the “predetermined directed energy therapy” for the subject 22.

In general, the treatment plan may include indications regions of the body where therapeutic energy is to be directed. Further, the treatment plan may also include a number of sessions to be administered, duration of each session, a time schedule of the sessions, time sequence of administration of directed energy, and intensities and frequencies of the energy. Furthermore, the treatment plan may also indicate one or more biometric signals to be monitored before, during or after administration of the treatment. Similarly, the treatment plan may also indicate one or more cognitive, behavioral, or physical tests to be administered to the subject 22 before, during or after administration of the treatment. Additionally, the treatment plan may indicate the manner in which current or subsequent administration of directed energy is to be controlled based on the one or more biometric signals and/or results of the cognitive, behavioral, or physical tests.

Subsequent to identifying the treatment plan, a directed energy emission device 24 may be placed proximate to a target area of the subject's body. The directed energy emission device 24 may include a plurality of energy-emitting portals 30 in communication with a source of directed energy 26. The source of directed energy 26 may be capable of emitting energy in the form of at least one of visible light, infrared light, near infrared light, electromagnetic fields (pulsed and static), ultrasound, and millimeter waves (or other suitable energy types), directed towards the target site. In some embodiments, the energy emission portals 30 may include a light emitting diode capable of emitting light with a wavelength of between 450 nanometers and 1500 nanometers (e.g. visible light, infrared light, and near infrared light) Further, in some embodiments, the energy emission portals 30 may include a distal end of a fiber optic transmission medium providing luminous communication from a source of light with a wavelength of between 450 nanometers and 1500 nanometers to the respective energy emission portal. Furthermore, the energy emission device 24 may itself include a source of light configured to emit energy including a wavelength of between 450 nanometers and 1500 nanometers. In either or both cases, the source of light may be any suitable element for generating light energy, including but not limited to a bulb and/or light emitting diode. It is also contemplated that the source of light may be a laser (fixed frequency or variable/tunable frequency) capable of delivering directed energy to the subject via the energy emission device 24, whether by having the laser source (not shown) as part of the energy emission device 26 or delivered to the energy emission device 26 via other means (e.g. fiber optics discussed above).

Additionally, the energy emission device 24 includes an energy source 26. In some embodiments, the energy source 26 may include multiple sources of energy generation and at least two of the sources of energy generation may be configured to generate energy in different wavelength bands. For example, different wavelength bands of light energy may be provided to include one band of light energy with a wavelength between 450 nanometers and 1000 nanometers and a second band of light energy with a wavelength greater than 1000 nanometers. As another example, it is also contemplated that different sources of directed energy may be used wherein each has a different frequency (e.g. near infrared energy at one frequency and magnetic energy at another frequency).

Further, the method may include a step of delivering one or more types of directed energy 32a, 32b, 32c, 32d from a specific selection of energy portals 30a, 30b, 30c, 30d for a predetermined period of time with a predetermined energy level. Accordingly, a controller 34 in logical communication with the source of directed energy 26 may be operative to cause the source of directed energy 26 to provide a predetermined directed-energy therapy to at least some of the energy emitting portals 30a-30d. Further, the specific selection of energy portals 30a-30d may include less than all of the energy emitting portals 30a-30d. The controller 34 may include a processor and digital data storage.

Additionally, the method may include a step quantifying one or more biometric measurements of the subject 22 using a biometric measurement device 36. In some embodiments, the biometric measurement device may include an electronic gaming device requiring inputs based upon cognitive analysis and manipulation of a user input mechanism. Further, in some embodiments, the biometric measurement device comprises an EEG apparatus (whether standard or quantitative/QEEG). In other embodiments, the biometric measurement device may be any that is capable of measuring skin tension, electrical conductivity, breath analysis, muscle tension and integrity, cell surface potential, pH, salt, and other chemical sensitivity. The biometric measurement device may be in logical communication with the controller 34 to provide digital data to the controller 34 indicative of the one or more biometric measurements from the subject 22 at least one of before, during and after the administration of the predetermined directed energy therapy.

Further, the method may include a step of selectively modifying the predetermined directed energy therapy to the subject 22 based on the one or more biometric measurements from the biometric measurement device. The modifying may include at least one of modifying the selection of the energy portals 30a-d, the type of directed energy 32a-d, the level of directed energy, the duration of the directed energy, and the frequency of the directed energy.

Accordingly, a source of electrical logic signals may be placed in logical communication with the controller 34 and capable of causing the controller 34 to receive input from the biometric measurement device and cause the source of directed energy 30 to continue delivering the predetermined directed energy therapy or selectively modify the predetermined directed energy therapy. Further, in some embodiments, the source of electrical logic signals may be placed in logical communication with the controller 34 including a digital storage storing executable code upon command.

In some embodiments, the multiple energy emitting portals 30a-30d may be provided in an emitter array 28 comprising a matrix of energy emitting portals 30a-30d that may be activated by the controller 34 to emit multiple forms of directed energy 32a-32d independent of other energy emitting portals 30a-30d. Additionally, in some embodiments, the multiple energy emitting portals 30a-30d including the matrix may be identifiable via any number of suitable coordinate systems, including not necessarily limited to, Cartesian, Polar, number line, cylindrical, spherical, homogenous, curvilinear, orthogonal, skew log-polar, Plucker, canonical, parallel, barycentric, trilinear, and any transformation thereof. Further, the controller 34 may activate one or more of the energy emitting portals 30a-30d according to a pattern that may be associated with multiple predetermined coordinates. It is also contemplated that the energy emitting portals 30a-30d may be located according to the internationally recognized 10/20, 10/10 and 10/5 protocol or methodology from commercially available brain-mapping systems.

In some embodiments, the biometric measurement device 36 may provide biometric measurements indicative of stimulation in the target tissue. In some embodiments, the biometric measurement device 36 may provide biometric measurements indicative of reduced activity in the target tissue.

In some embodiments, the controller 34 may identify a front portion and a back portion of the matrix and activate energy emitting portals 30 in a pattern generally beginning at the front portion of the matrix and continuing to the back portion of the matrix. In some embodiments, the controller 34 may identify a left portion and a right portion of the matrix and activate energy emitting portals 30 in a pattern generally beginning at the left portion of the matrix to the right portion of the matrix. In some embodiments, the controller 34 may identify a center portion and a periphery portion of the matrix and activate energy emitting portals 30 in a pattern generally beginning at the center portion of the matrix to the periphery portion of the matrix. In some embodiments, the controller 34 may activate energy emitting portals 30 in a stochastic pattern throughout the matrix.

In some embodiments, the biometric measurement device may include an electronic gaming device requiring inputs based upon cognitive analysis and manipulation of a user input mechanism. In some embodiments, the biometric measurement device 36 may include an elecroencephalography (EEG, whether standard or quantitative/QEEG) apparatus, an electromyography (EMG) apparatus, a thermometer, an electrodermography (EDG) apparatus, a photoplethysmography (PPG) apparatus, an electrocardiogram (ECG) apparatus, a pneumography apparatus, a capnography apparatus, a rheonocephalography (REG) apparatus, and/or a hemoencephalography (HEG) apparatus. In still other embodiments, the biometric measurement device 36 comprises any device or apparatus useful for measuring skin tension, electrical conductivity, breath analysis, muscle tension and integrity, cell surface potential, pH, salt, and/or other chemical sensitivity.

Furthermore, in some embodiments, the controller 34 may be additionally in logical communication with a digital communications network and the source of electrical logic signals may include a network access device communicating the electrical logic signals via the digital communications network. Further, the network access device may be configured to additionally receive the digital data indicative of the one or more biometric measurements.

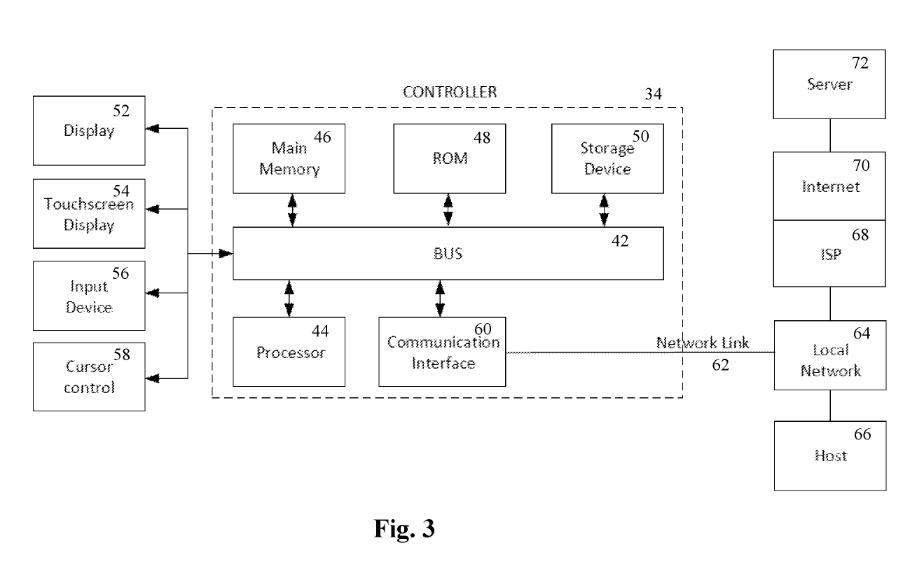

Referring now to FIG. 3, a block diagram of a controller configured for providing therapeutic administration of directed energy to a subject in accordance with some embodiments is illustrated. Additional aspects of controller hardware that may be included as computer hardware, useful for implementing the present disclosure, may be illustrated as a block diagram that may include a controller 34 upon which an embodiment of the invention may be implemented. Controller 34 may include a bus 42 or other communication mechanism for communicating information, and a processor 44 coupled with bus 42 for processing information.

Controller 34 may also include a main memory 46, such as a random access memory (RAM) or other dynamic storage device, coupled to bus 42 for storing information and instructions to be executed by processor 44. Main memory 46 may also be used for storing temporary variables or other intermediate information during execution of instructions to be executed by processor 44. Controller 34 may further include a read only memory (ROM) 48 or other static storage device 50.

Controller 34 may be coupled via bus 42 to a display 52, such as a liquid crystal display (LCD), for displaying information to a computer user. Alternatively, the controller 34 may be coupled to a touchscreen display 54 which enables a user to input data by making selections directly on the screen. An input device 56, including alphanumeric and other keys, or modes of input, such as, for example, a microphone and a radio frequency device such as Bluetooth, may be coupled to bus 42 for communicating information and command selections to processor 44. Another type of user input device may be a cursor control 58, such as a mouse, a trackball, a touchpad, touchscreen, or cursor direction keys for communicating direction information and command selections to processor 44 and for controlling cursor movement on display 52. This input device may typically have two or three degrees of freedom in two axes, a first axis (e.g., x) and a second axis (e.g., y), that allows the device to specify positions in a plane or stereo cameras that process and provide a third axis of input.

Some embodiments of the invention may be related to the use of controller 34 for setting operational parameters. According to one embodiment of the invention, control parameters may be defined and managed by controller 34 in response to processor 44 executing one or more sequences of one or more instructions contained in main memory 46. Such instructions may be read into main memory 46 from another computer-readable medium, such as storage device 50. Execution of the sequences of instructions contained in main memory 46 causes processor 44 to perform the process steps described herein. In alternative embodiments, hard-wired circuitry may be used in place of or in combination with software instructions to implement the invention. Thus, embodiments of the invention are not limited to any specific combination of hardware circuitry and software.

The term “computer-readable medium” as used herein may refer to any medium that participates in providing instructions to processor 44 for execution. Such a medium may take many forms, including but not limited to, non-volatile media, volatile media, and transmission media. Non-volatile media includes, for example, solid-state devices (SSD) or magnetic disks, such as storage device 50. Volatile media may include dynamic memory, such as main memory 46. Transmission media includes coaxial cables, copper wire and fiber optics, including the wires that comprise bus 42. Transmission media may also take the form of infrared and radio frequency transmissions, acoustic or light waves, such as those generated during radio wave and infrared data communications.

Common forms of computer-readable media may include, for example, a memory stick, hard disk or any other magnetic medium, an optical medium, a RAM, a PROM, and EPROM, a FLASH-EPROM, any other memory chip or cartridge, a carrier wave as described hereinafter, or any other medium from which a computer can read.

Various forms of computer readable media may be involved in carrying one or more sequences of one or more instructions to processor 44 for execution. For example, the instructions may initially be carried on a magnetic disk of a remote computer. The remote computer can load the instructions into its dynamic memory and send the instructions over a distributed network such as the Internet. A communication device may receive the data on the telephone line and use an infrared transmitter to convert the data to an infrared signal. An infrared detector may receive the data carried in the infrared signal and appropriate circuitry can place the data on bus 42. Bus 42 may carry the data, or otherwise be in logical communication to the main memory 46, from which processor 44 retrieves and executes the instructions. The instructions received by main memory 46 may optionally be stored on storage device 50 either before or after execution by processor 44.

Controller 34 may also include a communication interface 60 coupled to bus 42. Communication interface 60 provides a two-way data communication coupling to a network link 62 that may be connected to a local network 64. For example, communication interface 60 may operate according to the Internet protocol. As another example, communication interface 60 may be a local area network (LAN) card a data communication connection to a compatible LAN.

Network link 62 may typically provide data communication through one or more networks to other data devices. For example, network link 62 may provide a connection through local network 64 to a host computer 66 or to data equipment operated by an Internet Service Provider (ISP) 68. ISP 68 in turn may provide data communication services through the worldwide packet data communication network now commonly referred to as the “Internet” 70. Local network 64 and Internet 70 may both use electrical, electromagnetic or optical signals that carry digital data streams. The signals may be transmitted through the various networks and the signals on the network link 62 and through communication interface 60, which carry the digital data to and from controller 34 are exemplary forms of carrier waves transporting the information.

In some embodiments, Controller 34 may send messages and receive data, including program code, through the network(s), network link 62 and communication interface 60. In the Internet example, a server 72 might transmit a requested code for an application program through Internet 70, ISP 68, local network 64 and communication interface 60.

Processor 44 may execute the received code as it is received, and/or stored in storage device 50, or other non-volatile storage for later execution. Some exemplary controllers 34 may include a personal digital assistant, a mobile phone, a smart phone, a tablet, a netbook, a notebook computer, a laptop computer, a terminal, a kiosk or other type of automated apparatus. Additional exemplary devices may include any device with a processor executing programmable commands to accomplish the steps described herein.

A controller 34 may include one or more of: personal computers, laptops, pad devices, mobile phone devices and workstations located locally or at remote locations, but in communication with the controller. System apparatus may include digital electronic circuitry included within computer hardware, firmware, software, or in combinations thereof. Additionally, aspects of the invention may be implemented manually.

Apparatus of the disclosure may be implemented in a computer program product tangibly embodied in a machine-readable storage device for execution by a programmable processor and method actions can be performed by a programmable processor executing a program of instructions to perform functions of the invention by operating on input data and generating output. The present disclosure may be implemented advantageously in one or more computer programs that are executable on a programmable system including at least one programmable processor coupled to receive data and instructions from, and to transmit data and instructions to, a data storage system, at least one input device, and at least one output device. Each computer program may be implemented in a high-level procedural or object oriented programming language, or in assembly or machine language if desired, and in any case, the language can be a compiled or interpreted language. Suitable processors may include, by way of example, both general and special purpose microprocessors.

Generally, a processor may receive instructions and data from a read-only memory and/or a random access memory. Generally, a computer may include one or more mass storage devices for storing data files; such devices include Solid State Disk (SSD), magnetic disks, such as internal hard disks and removable disks magneto-optical disks and optical disks. Storage devices suitable for tangibly embodying computer program instructions and data include all forms of non-volatile memory, including, by way of example, semiconductor memory devices, such as EPROM, EEPROM, and flash memory devices; magnetic disks such as, internal hard disks and removable disks; magneto-optical disks; and CD_ROM disks may be included. Any of the foregoing may be supplemented by, or incorporated in, ASICs (application-specific integrated circuits).

In some embodiments, implementation of the features of the present disclosure may be accomplished via digital computer utilizing uniquely defined controlling logic, wherein the controller includes an integrated network between and among the various participants in Process Instruments.

The specific hardware configuration used may not be particularly critical, as long as the processing power is adequate in terms of memory, information updating, order execution, redemption and issuance. Any number of commercially available database engines may allow for substantial account coverage and expansion. The controlling logic may use a language and compiler consistent with that on a CPU included in the controller. These selections may be set according to per se well-known conventions in the software community.

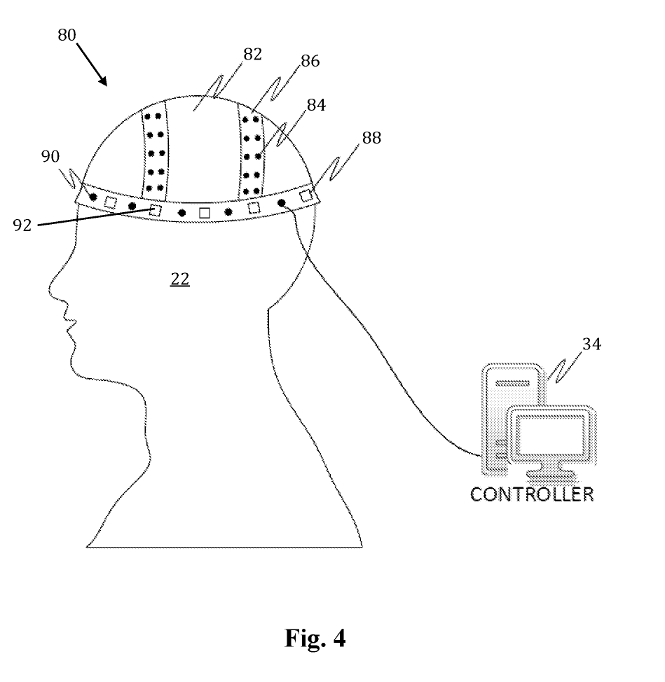

Referring now to FIG. 4, in certain cranial directed energy implementations, a cranial directed energy emission device 80 may include a harness, straps, skull cap, helmet, cap, band or other support structure suitable for positioning energy emitting portals 84 proximate to a surface area of a subject's skull 82. The cranial directed energy emission device 80 is in logical communication with a controller 34. The energy emitting portals 84 are positioned to direct emitted energy towards a cranium such that the directed energy may provide therapeutic impact on the skull (that is, the bone and constituents thereof such as red blood cells) as well as the underlying neuronal and other cellular constituents of the brain within the skull. In some embodiments, energy emitting portals 84 may be arranged in an array 86 wherein respective energy emitting portals 84 may be selected for transmission of directed energy at a particular instance of time relative to a treatment profile. Positioning of the energy emitting portals 84 proximate to the subject's skull 82 may if preferable be accomplished via fastening techniques allowing for removal with relative ease, while at the same time securing the energy emitting portals 84 relative to portions of the cranium of the subject during administration of a treatment session. Fastening techniques may therefore include, by way of non-limiting example: Velcro™, elastic constraints, stretchable fabric, a hard shell skull cap, a soft shell skull cap, an adjustable harness, and the like.

In another aspect, the cranial directed energy emission device 80 may include a plurality of energy-emitting portals 84 that incorporate a source of directed energy within the portal itself, such as, for example one or more light-emitting diode capable of generating and directing at least one of visible light, infrared light, near infrared light to the subject according to an aspect of the invention. Other implementations include a plurality of energy-emitting portals 84 in the form of fiber optic elements in luminous communication with a source of directed energy 88 capable of generating any of a variety of energy types suitable for transmission through the fiber optic elements to be directed at or towards the subject. Suitable energy types for fiber optic transmission may include, but are not necessarily limited to, visible light, infrared light and near infrared light, which may be produced by any number of suitable light sources such as (by way of non-limiting example) LEDs, laser emitting diodes, and any other source of laser energy. The source of directed energy 88 may be capable of emitting energy in the form compatible with a predetermined treatment profile. Forms of directed energy may include, by way of non-limiting example, one or more of: visible light, infrared light, near infrared light, electromagnetic fields, ultrasound, and millimeter waves. In still another aspect, directed energy emission device 80 may include one or more biometric measurement devices 90 operable to perform one or more of measurements quantifying biological aspects of the subject, as described above.

In another aspect, the cranial directed energy emission device 80 comprises a multi-modal cranial appliance capable of performing multiple functions (in this case three), including (a) pre-therapy assessment (e.g. standard or quantitative EEG); (b) directed energy therapy (one or more of the types of directed energy disclosed herein or later developed); and (c) neurofeedback (e.g. EEG with Loretta Z-score neurofeedback based on audio stimulation, visual stimulation, trans-cranial electrical stimulation, and/or suitable forms of electromagnetic stimulation). To facilitate the pre-therapy assessment and/or neurofeedback functions, the cranial directed energy emission device 80 may be further provided with a pair of electrodes 92 positioned (by way of example only) at any of a variety of desired locations proximate to the subject's cranium (e.g. on either side of the subject's 22 forehead) when the subject 22 is wearing the cranial directed energy emission device 80. The electrodes 92 may be any of a variety of components suitable to direct energy towards the subject's cranium, and in some embodiments may also be capable of receiving signals from the subject (e.g. EEG, whether standard or quantitative), including but not limited to dry electrodes, wet electrodes and traditional electrodes. If electrodes 92 are provided as “dual purpose” electrodes (both sending directed energy and receiving signals), then the electrodes 92 may be capable of performing directed energy therapy along with pre-therapy assessment and/or neurofeedback, according to an aspect of the invention. In this fashion, it is possible to have multiple modalities (in this case three) in a single wearable cranial appliance, which increases efficiency in the therapy session and enables the directed energy therapy to be modified or continue unchanged in response to neurofeedback received during the session. It will be appreciated that the multi-modal cranial appliance may also be equipped with electrodes that have a singular function (e.g. only for sending directed energy or only for receiving signals) depending upon the type of directed energy to be administered by the multi-modal cranial appliance and the various types of signals that are desired to be received (e.g. from one or more of the biometric measurement devices disclosed herein or later developed). The multi-modal cranial appliance may also include any of a variety of devices or systems for heating or cooling the subject's cranium during use.

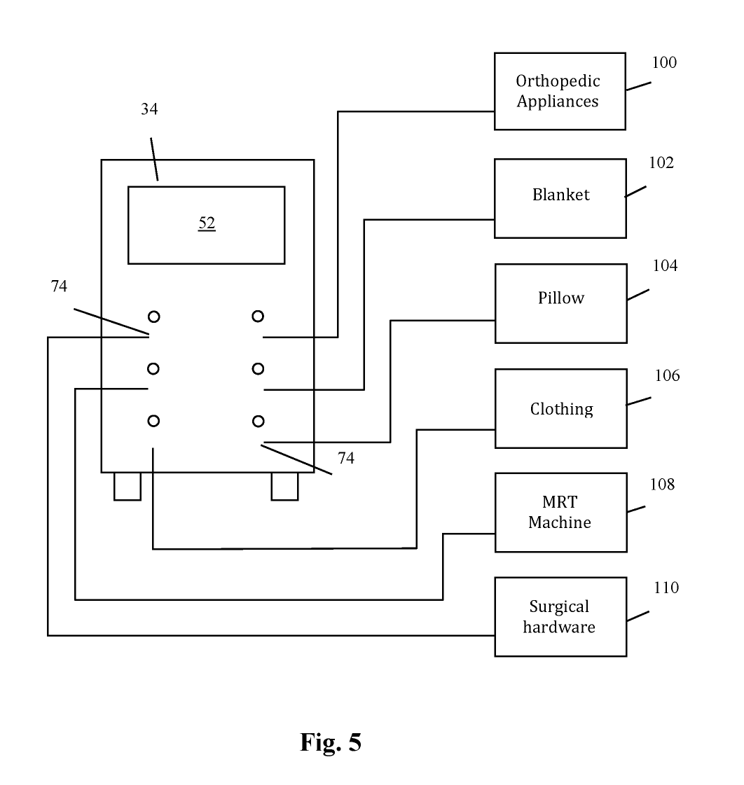

FIG. 5 is a diagram illustrating various non-cranial applications of directed energy according to other aspects of the present invention. In addition to head mounted apparatus described above, directed energy capability may be added to many different articles to deliver therapeutic directed energy to targeted areas. Each of these articles may be placed in electrical communication with the controller 34, either through a hard-wired connection or wirelessly, such that the controller 34 is able to control the predetermined directed energy therapy protocol. Controller 34 includes a display 52 and a variety of input ports 74. The input ports 74 are configured to receive a wired connection with various articles that have been modified to deliver directed energy therapy to a target area on a subject 22. Examples of these articles, which will be discussed in further detail below, include orthopedic appliances 100, blanket 102, pillow 104, clothing 106, a multiple resonance therapy (MRT) system 108, and other surgical hardware 110.

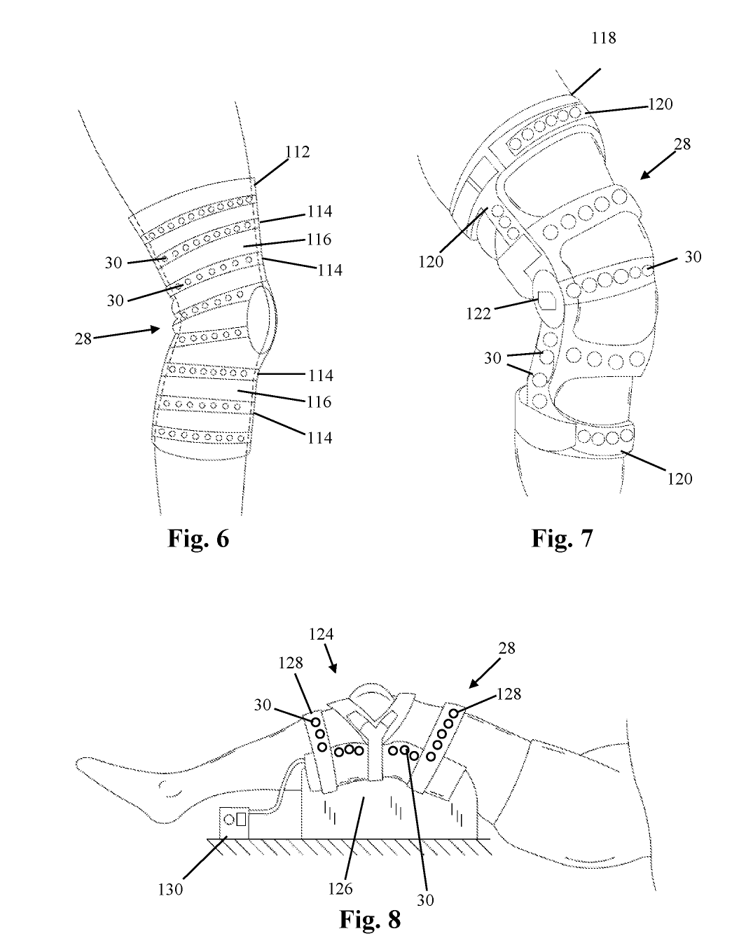

FIGS. 6-10 illustrate several examples of orthopedic appliances 100 that have been adapted to deliver therapeutic directed energy to targeted areas, which are shown and described by way of illustration only, and do not represent an exclusive set. In each case, the various orthopedic appliances include a directed energy emitter array 28, including a plurality of directed energy emitters or portals 30 distributed strategically throughout the appliance. The emitters 30 are operable such that any number of them may be activated at any one time, and the combination of active emitters may be selectively modified to change the location of the applied directed energy and thereby optimize the treatment. The emitters 30 may be configured such that they are all the same, and emit the same type of directed energy 32a. Alternatively, the plurality of emitters 30 may include a mixture of different types of emitters 30a-30d, with each type of emitter configured to emit a different type of directed energy 32a-32d. In this fashion, a user (e.g. physician, therapist, caregiver, patient, etc. . . . ) would be able to change the type of directed energy applied to a specific target site without having to change the appliance.

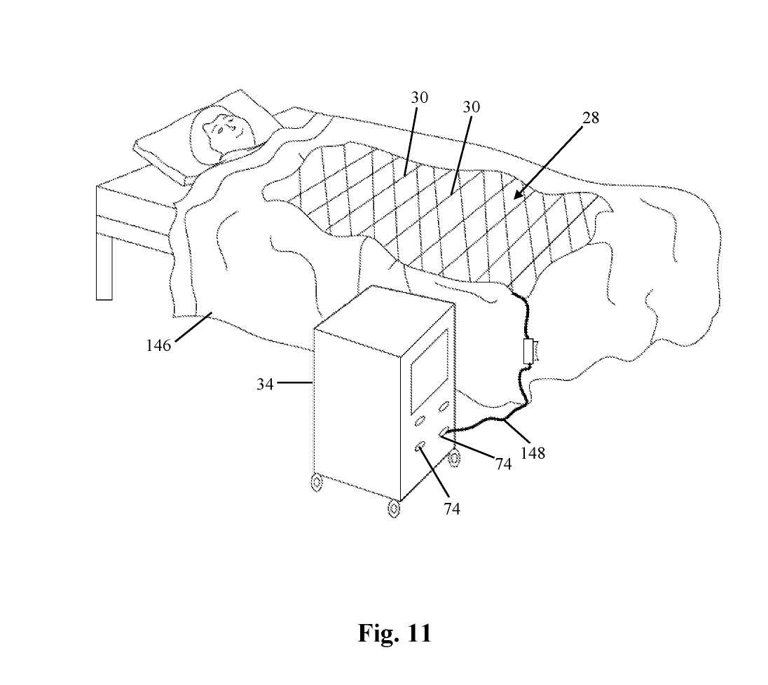

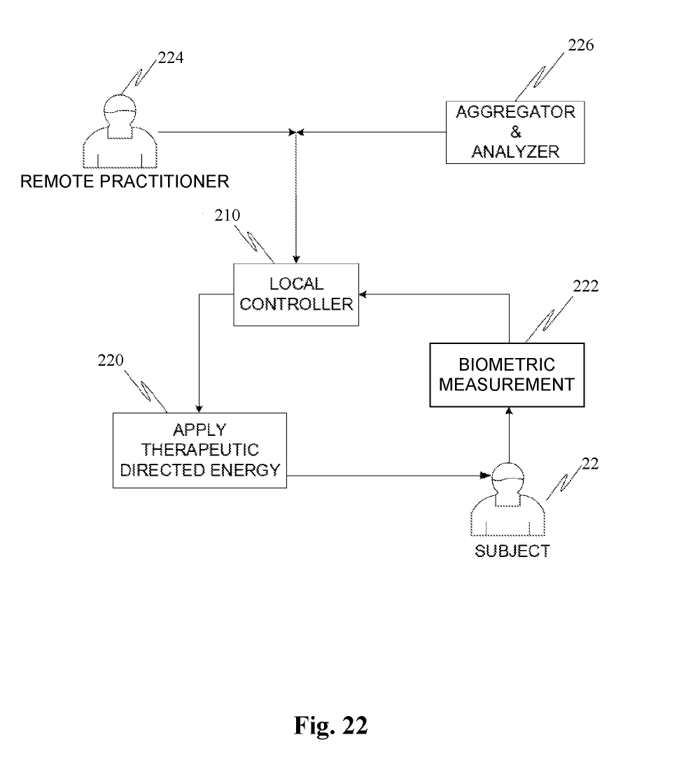

In each case, the directed energy emitter array 28 is programmed to emit a particular type of energy to a particular targeted location at a specific intensity for a predetermined length of time. Each of these factors may be variable during treatment, and controlled by a controller 34 that may be operated manually by the user or automatically by a predetermined program. The controller may be connected to each appliance via a wire (e.g. FIG. 11) or via a wireless connection (e.g. FIG. 22).

FIG. 6 illustrates an example of a flexible knee brace 112 modified to include a directed energy emitter array 28. By way of example, the knee brace 112 may be made of any material commonly used (e.g. neoprene, etc). The directed energy emitter array 28 in the example shown includes a plurality of emitters 30 arranged in linear fashion along a plurality of circumferential bands 114. The bands 114 are distributed uniformly throughout the knee brace 112 with a spacing 116 between them. This arrangement is shown by way of example only and it should be noted that the emitters 30 may be provided on the knee brace 112 in any number, combination, and distribution pattern, up to and including a continuous matrix of emitters 30 with no significant space between them. The emitters 30 may be all one type (as shown by way of example) or a combination of different types of emitters adapted to emit different types of directed energy. Since the flexible knee brace 112 is at its core a standard knee brace, the patient gets all the benefit of wearing the knee brace as well as the benefit of therapeutic directed energy treatment, for example including but not limited to increased mitochondrial ATP production, increased stem cell production, decreased inflammation, increased blood flow, and opiate production.

FIG. 7 illustrates an alternative example of a knee brace 118, which would be used in instances where the wearer is in need of greater stability of the knee. The knee brace 118 of the present example includes several rigid bar components 120 that provide stability and a hinge mechanism 122 that allows the knee to bend. The knee brace 118 is modified to include a directed energy emitter array 28. The directed energy emitter array 28 includes a plurality of directed energy emitters 30 distributed along the rigid bar components of the brace. The emitters 30 may be all one type (as shown by way of example) or a combination of different types of emitters adapted to emit different types of directed energy.

FIG. 8 illustrates an example of another type of knee appliance 124 that is modified to provide directed energy therapy. The knee appliance 124 of the instant example includes a base 126 that is configured to cradle the subject's knee as the subject lies in a prone position. The knee appliance 124 further includes a plurality of straps 128 to secure the subject's knee to the base 126. The knee brace includes a directed energy appliance 28, including a plurality of directed energy emitters 30 distributed along the base 126 and strap 128 components of the appliance 124. The emitters 30 may be all one type (as shown by way of example) or a combination of different types of emitters adapted to emit different types of directed energy. Also shown by way of example is an energy source 130 that is in communication with the emitter array 28.

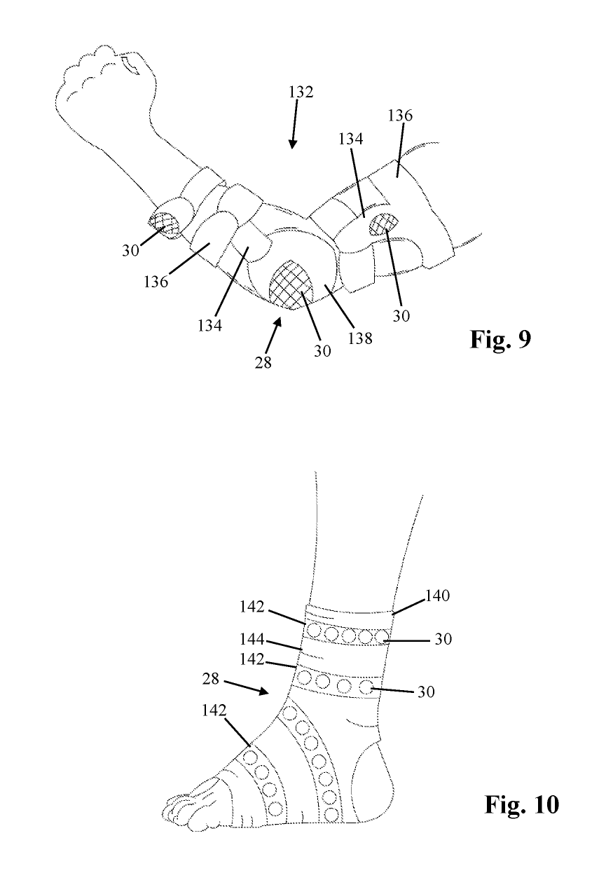

FIG. 9 illustrates an example of an arm brace 132 that has been modified to include a directed energy emitter array 28 according to another embodiment. The arm brace 132 generally comprises a plurality of stability elements 134 and securing straps 136 as well as an elbow pad 138. Directed energy emitters 30 may be distributed along any portion of the elbow brace 132 and are thus are capable of delivering energy to target areas in the upper arm, forearm and/or elbow.

FIG. 10 illustrates an example of an ankle sleeve 140 that has been modified to include a directed energy emitter array 28 according to another embodiment. By way of example, the ankle brace 140 may be made of any material commonly used (e.g. neoprene, etc). The directed energy emitter array 28 in the example shown includes a plurality of emitters 30 arranged in linear fashion along a plurality of circumferential bands 142. The bands 142 are distributed uniformly throughout the ankle brace 140 with spacing 144 between them. This arrangement is shown by way of example only and it should be noted that the emitters 30 may be provided on the ankle brace 140 in any number and in any distribution pattern, up to and including a continuous matrix of emitters with no significant space between them.

The various orthopedic appliances described in the preceding examples are each used to treat or help rehabilitate an injured body part, or one in which the patient is experiencing pain, soreness, etc. Thus, the recovery benefits that a patient receives from using the particular appliances in the first place are further enhanced with the therapeutic benefits of an applied directed energy protocol. Such benefits realized by the patient may include faster healing/recovery due to increased ATP production at the cellular level as well as reduced pain due to opiate (endorphin) production, production of endogenous stem cells, and a reduction of inflammation.

In some instances, it may be desirable to pursue a directed energy therapy that targets different locations distributed throughout the entire body. FIG. 11 illustrates a blanket 146 that has been equipped with a directed energy emitter array 28. In the instant example, the directed energy emitter array 28 comprises a section of the blanket 146 that is at least large enough to cover an entire human body. The directed energy emitter array 28 includes a plurality of directed energy emitters 30 distributed strategically throughout the blanket 146. By way of example, the present emitters 30 are provided along a series of crosshatched filaments, however other forms and distributions are possible. The emitters 30 are operable such that any number of emitters 30 may be activated at any one time, and the combination of active emitters 30 may be changed to change the location of the applied directed energy and thereby optimize the therapy. The emitters 30 may be configured such that they are all the same, and emit the same type of directed energy. Alternatively, the emitters 30 may be configured to emit multiple forms of directed energy. As a further alternative, the plurality of emitters 30 may include a mixture of different types of emitters, with each type of emitter configured to emit a different type of directed energy. In this fashion, a user would be able to change the type of directed energy applied to a specific target site without having to change the blanket.

In the example shown in FIG. 11, the blanket 146 is placed over a user lying in a bed. The blanket 146 is connected via a wire 148 to a controller 34, for example the controller shown in FIG. 5. The controller 34 includes a user interface screen 52 and a plurality of input jacks 74. The user interface screen 52 is programmable to administer a specified predetermined therapy protocol, either via a touch-screen technology or via an attached keyboard (not shown). The input jacks 74 may be configured to receive plug attachments for various different directed energy treatment devices to be used in conjunction with the blanket 146 to achieve optimal therapy.

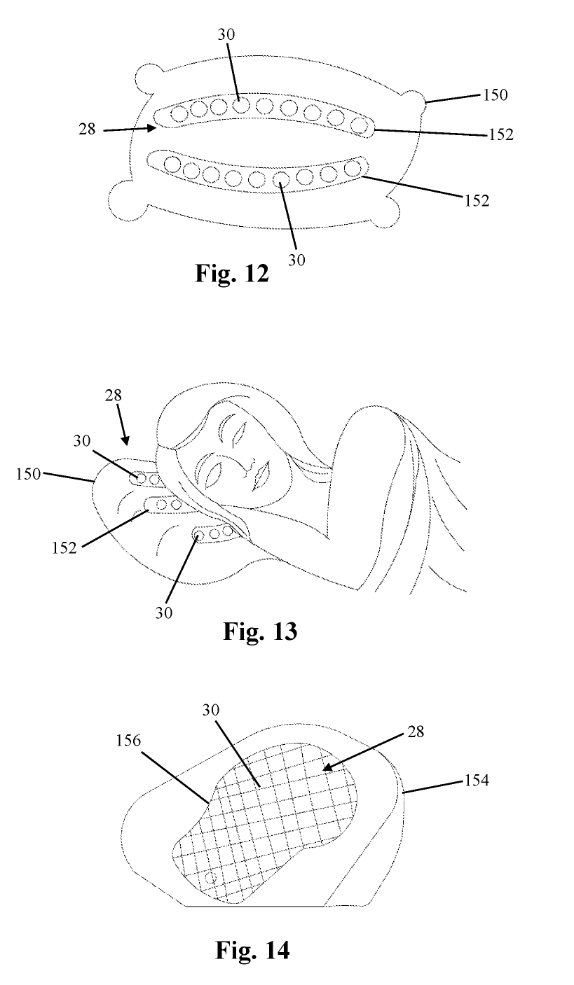

FIGS. 12-13 illustrates an example of a pillow 150 that has been modified to include a directed energy emitter array 28 according to another embodiment. The directed energy emitter array 28 in the example shown includes a plurality of emitters 30 arranged in a generally linear fashion in bands 152 extending along the surface of the pillow 150. The bands 152 are spaced evenly apart and are configured to position at least a few of the emitters at strategic places on the user's head when the user is using the pillow (e.g. FIG. 13). This arrangement is shown by way of example only and it should be noted that the emitters 30 may be provided on the pillow 150 in any number and in any distribution pattern, up to and including a continuous matrix of emitters with no significant space between them. The emitters 30 are operable such that any number of emitters 30 may be activated at any one time, and the combination of active emitters 30 may be changed to change the location of the applied directed energy and thereby optimize the therapy. The emitters 30 may be configured such that they are all the same, and emit the same type of directed energy. Alternatively, the emitters 30 may be configured to emit multiple forms of directed energy. As a further alternative, the plurality of emitters 30 may include a mixture of different types of emitters, with each type of emitter configured to emit a different type of directed energy. In this fashion, a user (or therapist) would be able to change the type of directed energy applied to a specific target site without having to change the pillow 150. As with previous examples, the pillow 150 is in communication with a controller (not shown), either via a wired or wireless connection.

FIG. 14 illustrates an alternative example of a pillow 154 modified to include a directed energy emitter array 28. The pillow 154 by way of example is a form-fitting pillow that has a formed cavity 156 for receiving a patient's head therein. The formed cavity 156 is equipped with a directed energy emitter array 28. The array 28 comprises a plurality of directed energy emitters 30 distributed throughout the cavity 156. The form-fitted pillow 154 may allow for a more precise application of directed energy compared with a standard pillow as described above with reference to FIG. 13, in that it designed to restrict movement of the patient's head while sleeping.

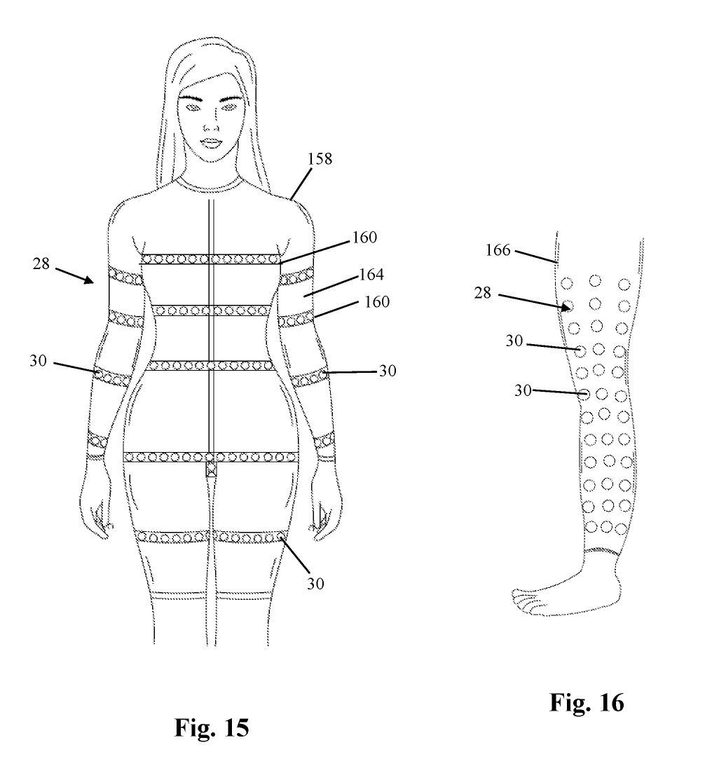

FIG. 15 illustrates an example of clothing 158 that has been modified to include a directed energy emitter array 28. By way of example, the emitter array 28 includes a plurality of emitters 30 arranged in linear fashion along a plurality of circumferential bands 160. The bands 160 are distributed uniformly throughout the clothing 158 with spacing 164 between them. This arrangement is shown by way of example only and it should be noted that the emitters 30 may be provided on the clothing 158 in any number and in any distribution pattern, up to and including a continuous matrix of emitters with no significant space between them. Although shown by example herein as a unitary article of clothing, it should be understood that any form of clothing that one normally wears may be modified to add an directed energy emitter array 28, for example including but not limited to shirts, pants, sweatshirts, shorts, under garments (e.g. to treat sexual dysfunction such as erectile dysfunction), vests, and the like.

FIG. 16 illustrates a legging 166 forming part of an article of clothing that includes a directed energy emitter array 28. In the instant example, the emitter array 28 comprises a plurality of directed energy emitters 30 distributed uniformly throughout the legging 166. The legging 166 can be part of a larger piece of clothing such as the body suit described in FIG. 15, or the legging could be a standalone article of clothing.

According to another embodiment of this disclosure, a variety of directed energy therapies may be delivered concurrently to treat several different maladies at the same time. This multiple resonance therapeutic (MRT) system may be realized using a standalone dedicated MRT machine or by modifying existing devices that are already being utilized to obtain diagnostic information or perform a different kind of medical treatment (e.g. Mill, CT scan, PET scan, diathermy machine, rTMS, EEG, QEEG, blood pressure analyzer, heart flow output analyzer, oxygen sensors, pressure sensors, and the like) to also include directed energy capability with biofeedback to ensure the best treatment possible.

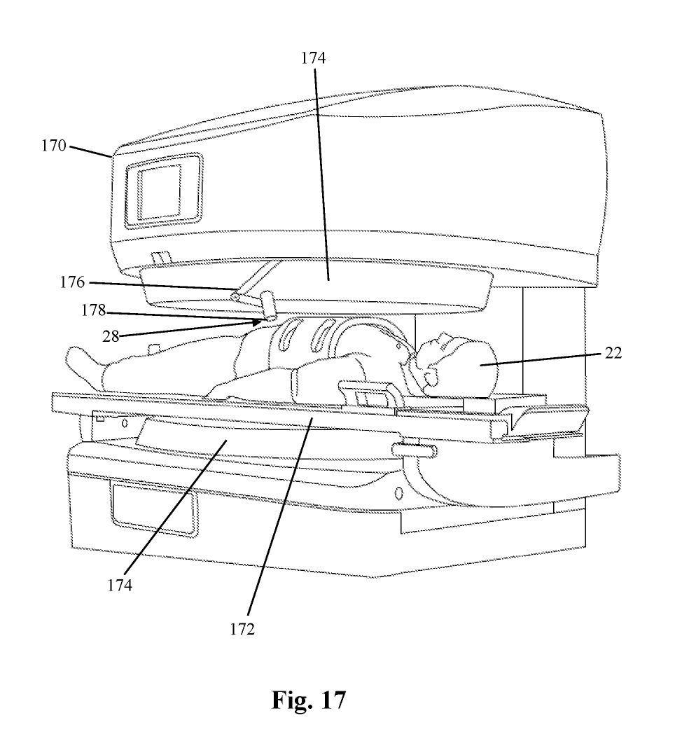

FIG. 17 illustrates an example of an open MM machine 170 that has been modified to include directed energy therapeutic capability. The modified open Mill machine 170 includes a patient table 172 and the imaging apparatus 174. The modified open MM machine 170 further includes a number of robotic arms 176 with an attached directed energy emitter array 28. As used in the MRT embodiments, a directed energy “emitter” is synonymous with the use of directed energy “portal” as described above, namely is may include any sutiable components for delivering directed energy to a desired anatomical location or region of a subject. For simplicity, the example shown in FIG. 17 includes only one such robotic arm 176, however it should be understood that any number of robotic arms 176 may be present. The directed energy emitter array 28 includes a distal surface 178 that further includes a plurality of directed energy emitters capable of emitting any form of directed energy discussed in this disclosure. The maneuverability of the robotic arms enable a user/programmer to position a number of directed energy emitters in any desired target area. For example, the use of multiple robotic arms 176 to deliver directed energy treatment allows such treatment to occur simultaneously at different target areas. Not only can the MRT machine 170 deliver directed energy treatment to multiple target areas simultaneously, but it can also deliver different directed energy treatment protocols to different target areas at the same time. Thus, a number of different maladies may be treated at the same time (if necessary).

There are several advantages to combining directed energy therapy with another medical procedure such as a MM. As discussed previously, the therapeutic benefits of directed energy treatment include pain reduction (via opiate production), increased blood flow (via vasodilation), faster healing (ATP production), and generation of new tissue (endogenous stem cell generation). Additionally, combining directed energy treatment therapy with a procedure such as a MRI exam reduces cost to the hospital and patient, reduces the time and inconvenience to the patient.

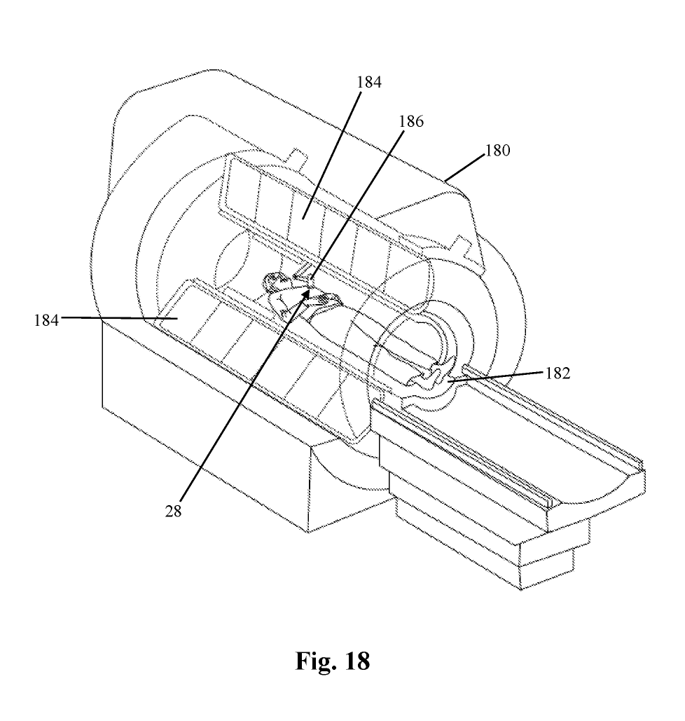

FIG. 18 illustrates another example of a MRT machine in the form of a full-body MM machine 180 that has been modified to include directed energy therapeutic capability. As with the open MM machine 170 discussed above, the modified full body MRI machine 180 includes a patient table 182 and the imaging apparatus 184. The modified full-body MRI machine 180 further includes a number of robotic arms 186 with an attached directed energy emitter array 28. For simplicity, the example shown in FIG. 18 includes only one such robotic arm 186, however it should be understood that any number of robotic arms 186 may be present. The robotic arms 186 of the present example are identical to the robotic arms 176 of the previously described example, thus although the structure is not easily viewable in FIG. 18, it should be understood that the directed energy emitter array 28 of the present robotic arm 186 also includes a distal surface that further includes a plurality of directed energy emitters capable of emitting any form of directed energy discussed in this disclosure. As described with the modified open Mill machine 170, the maneuverability of the robotic arms enable a user/programmer to position a number of directed energy emitters in any number of desired target areas to treat different areas at the same time.

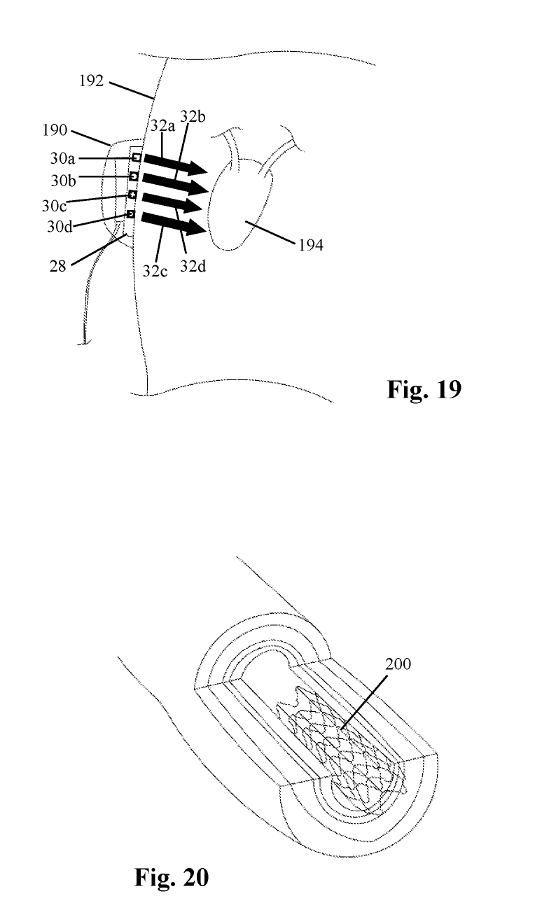

FIG. 19 illustrates an example of another externally applied apparatus 190 for administering directed energy therapy to a single location to achieve a variety of beneficial results. For example, the apparatus 190 of the present example may be placed on the subject's skin 192 directly over an organ such as a heart 194. The apparatus 190 is equipped with a direct energy emitter array 28 as described herein, with a plurality of direct energy emitters 30a-30d distributed thereon. Each of the direct energy emitters 30a-30d is configured to emit a different type of energy 32a-32d.

While multiple embodiments involve external administration of directed energy (that is, from emitters or portals located outside the subject), it is also contemplated to provide directed energy internal to the patient. For example, according to another embodiment of the disclosure, directed energy therapies (single or multiple) may be used to augment or enhance surgical procedures to impart the benefits of directed energy treatment to surgical target sites. For example, FIG. 20 illustrates an example of a stent 200 implanted into an artery. The stent 200 is adapted to deliver directed energy therapy to the inside of the body. To accomplish this, the stent 200 may be equipped with a plurality of tiny directed energy emitters embedded within the stent structure. The emitters may be powered while in direct contact with a source (for example during insertion) or the emitters may be activated by a wireless application of energy to the stent itself during treatment after insertion. In a similar fashion, other surgical implants and instruments may be modified to include direct energy emission capabilities. If used during a surgical procedure, for example using a special probe, the surgeon would be able to use directed energy to increase ATP in the tissue that the surgeon is addressing (or cutting through/away) to aid in faster healing, reduce pain, lower inflammation, induce vasoconstriction (for example to reduce blood flow and consequent blood loss), and simulate stem cell generation. These characteristics would be useful not only during surgery but after as well if the surgical implants that are left inside were also capable of being activated to deliver directed energy treatment to the surrounding tissue.

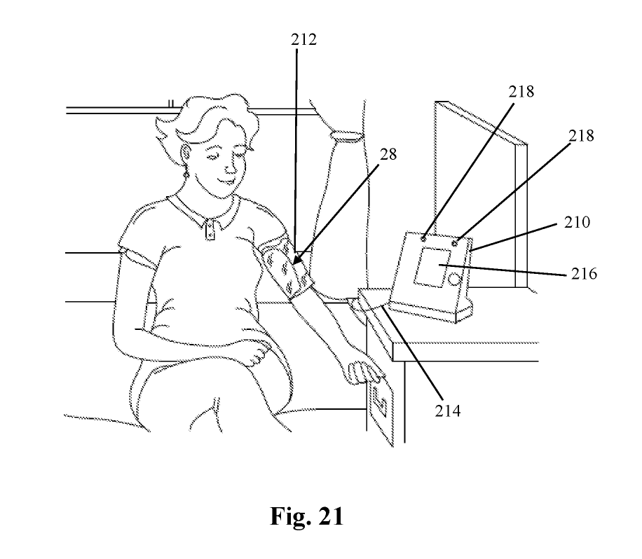

FIG. 21 illustrates an example of a portable controller 210 suitable for use in a patient's home. In the instant example, the portable controller 210 is connected to an orthopedic appliance (for example arm wrap 212) via a wire 214. However, a wireless connection is also possible (e.g. WIFI, Bluetooth, etc). As with previous examples described above, the arm wrap 212 includes a directed energy emitter array 28 attached to or incorporated within the wrap 212. The directed energy emitter array 28 is of the type described previously. The portable controller has a graphic user interface (GUI) 216 in the form of a screen that is programmable to communicate information to a user. The portable controller 210 may have additional auxiliary jacks 218 for connecting one or more types of directed energy sources with the given appliance (in this example, arm band 212) as well asone or more additional directed energy appliance (e.g. the cranial device in FIG. 4, the orthopedic appliances of FIGS. 6-10, the blanket of FIG. 11, the pillows of FIGS. 12-14, the clothing of FIGS. 15-16, and the handheld directed energy system for apparatus 190 of FIG. 19).

FIG. 22 is a block diagram illustrating the basic flow of information during a home directed energy therapy session, wherein the portable controller 210 of FIG. 21 is part of a remote or telemedicine system involving a remotely located practitioner (e.g. physician, therapist, nurse, etc. . . . ). The portable controller 210 is programmable to conduct directed energy therapy sessions while the subject 22 is in the comfort of their own home. The portable controller 210 executes the programmed therapeutic directed energy session 220 as selected by the remote practitioner. The portable controller 210 receives and records data associated with the directed energy therapy session, including duration, location, and intensity type(s) of energy applied, and also any biometric feedback 222 from the session. If the portable controller 210 is connected to a data network (e.g. cellular or the Internet), the collected data is then sent to the remote practitioner 224 (e.g. physician), who then analyzes the data and can remotely reprogram the portable control center 210 to change any aspect of the directed energy therapy protocol.

The portable controller 210 may also be capable of transmitting the data collected in near real time to a remote location over a data network such that a remote practitioner 224 monitoring the session remotely can adjust the directed energy therapy protocol while the treatment is occurring. If the portable controller 210 is not connected to a data network either during or after the session, then the collected data is stored within the controller 210. In such a case the portable controller 210 would have to be physically transported to the practitioner (or to a location where it can connect to a data network) in order to allow the therapist to analyze the collected data and alter the therapeutic protocol, if necessary. The local controller 210 may also be capable of transmitting the data to a centralized aggregator and analyzer server 226 even if the server 226 is in a different location than the remote practitioner.

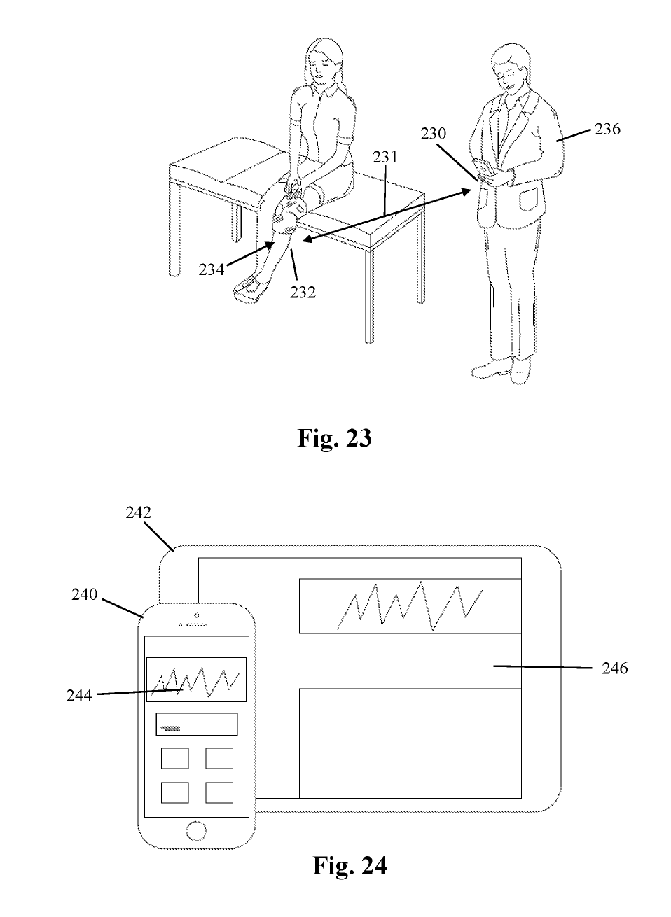

FIG. 23 illustrates an example of wireless communication between a handheld controller 230 and an example modified knee brace 232. As with previous examples, the modified knee brace 232 includes a directed energy emitter array 234 attached to or embedded within the knee brace 232. The directed energy emitter array 234 is of the type described previously herein. The handheld controller 230 is able to communicate 231 with the modified knee brace 232 wirelessly and in real time so the practitioner 236 may monitor and analyze the therapy session as it is occurring. In response to various biometric feedback indicators that the practitioner 236 may observe, the practitioner may be able to alter the type or combination of energy, intensity, duration, and location of the directed energy treatment as it is being administered. The system could also automatically adjust the directed energy therapy based on the biometric measurements (vs. having the practitioner do it manually) as previously described.

FIG. 24 illustrates several examples of handheld controllers such as a smart phone 240 and a tablet 242. The smart phone 240 and tablet 242 each have a graphic user interface, guis 244, 246, respectively that presents information relevant to the programmed therapeutic directed energy treatment. The GUIs 244, 246 also have input capability to receive instructions from a practitioner and communicate those instructions to the associated direct energy emitter device. The smart phone 240 and tablet 242 are able to communicate with directed energy emitter arrays (not shown) via any suitable wireless connection, including but not limited to Wi-Fi and Bluetooth. The smart phone 240 and tablet 242 are also configured to receive diagnostic and biometric feedback information during the treatment. A therapist holding smart phone 240 and/or tablet 242 is then able to monitor the treatment in real time and make adjustments based at least in part on this biometric feedback information, if necessary.

Certain features that are described in this specification in the context of separate embodiments can also be implemented in combination in a single embodiment. Conversely, various features that are described in the context of a single embodiment can also be implemented in combination in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous.

Moreover, the separation of various system components in the embodiments described above should not be understood as requiring such separation in all embodiments, and it should be understood that the described program components and systems can generally be integrated together in a single software product or packaged into multiple software products.

Thus, particular embodiments of the subject matter have been described. Other embodiments are within the scope of the following claims. In some cases, the actions recited in the claims can be performed in a different order and still achieve desirable results. In addition, the processes depicted in the accompanying figures do not necessarily require the particular order show, or sequential order, to achieve desirable results. In certain implementations, multitasking and parallel processing may be advantageous. Nevertheless, it will be understood that various modifications may be made without departing from the spirit and scope of the claimed disclosure.

What is claimed is:

1. Apparatus for providing directed energy therapy to a mammalian subject, the apparatus comprising:
a directed energy emission device positionable proximate to a surface of a region of interest of the mammalian subject, said directed energy emission device having a plurality of energy-emitting portals;
a source of directed energy in communication with said plurality of energy-emitting portals and capable of emitting energy in a form of at least one of visible light, infrared light, near infrared light, electromagnetic fields, ultrasound, and millimeter waves directed towards the region of interest via the energy-emitting portals of the directed energy emission device;
a controller in logical communication with the source of directed energy and operative to cause the source of directed energy to provide a predetermined directed energy therapy to at least some of the energy-emitting portals, wherein the predetermined directed energy therapy comprises delivering at least one type of directed energy from a specific selection of energy-emitting portals for a predetermined period of time with a predetermined energy level, said controller comprising a processor and a digital data storage;
a biometric measurement device capable of quantifying one or more biological aspects of the mammalian subject, the biometric measurement device in logical communication with the controller at least one of constantly and intermittently to provide digital data to the controller indicative of the one or more measurements quantifying biological aspects of the mammalian subject at least one of before, during, and after an administration of the predetermined directed energy therapy, said digital data for use by at least one of: (i) a neurofeedback system in logical communication with the controller including external stimulation administered to the mammalian subject in the form of at least one of visual stimulation, audio stimulation, trans-cranial electrical stimulation, trans-cranial magnetic stimulation, trans-tongue electrical stimulation, and trans-dermal electrical stimulation; (ii) a biofeedback system in logical communication with the source of directed energy to at least one of continue and modify at least one of the predetermined directed energy therapy and neurofeedback administered to the mammalian subject based on the one or more measurements quantifying biological aspects of the mammalian subject; and (iii) a data storage device for storing the one or more measurements quantifying biological aspects from the mammalian subject at least one of before, during, and after at least one of the predetermined directed energy therapy and neurofeedback therapy administered to the mammalian subject; and
a source of electrical logic signals in logical communication with the controller and capable of causing the controller to receive input from the biometric measurement device and cause the source of directed energy to continue delivering the predetermined directed energy therapy or selectively modify the predetermined directed energy therapy to the mammalian subject based on the input from the biometric measurement device, wherein modifying the predetermined directed energy therapy includes at least one of modifying the selection of the energy portals, the type of directed energy, the level of directed energy, the duration of the directed energy, and the frequency of the directed energy.

2. The apparatus of claim 1, wherein the controller is additionally in logical communication with a digital communications network and the source of electrical logic signals includes at least one network access device communicating the electrical logic signals via the digital communications network to deliver the predetermined directed energy therapy to the mammalian subject at a location remote from said controller.

3. The apparatus of claim 2, wherein the network access device additionally receives and delivers the digital data indicative of the feedback from the biometric measurement device to said controller at a location remote from said mammalian subject.

4. The apparatus of claim 1, wherein the energy emission portals comprise at least one of: (a) a plurality of light sources capable of emitting light with a wavelength of between 450 nanometers and 1500 nanometers, wherein said light sources include at least one of light emitting diodes and laser diodes; (b) a plurality of fiber optic transmission elements each having a distal end for providing luminous communication from a source of light with a wavelength of between 450 nanometers and 1500 nanometers to the respective energy emission portal, wherein said source of light includes at least one of a light emitting diode, a laser diode, a fixed frequency laser, and a variable frequency laser; (c) one or more ultrasound transducers; (d) one or more millimeter wave transducers; (e) one or more electrodes for delivering at least one of pulsed direct current (DC) and alternating current (AC); (f) one or more electromagnetic coils capable of emitting at least one of pulsed and static electromagnetic fields; and (g) combinational transducers capable of delivering any combination of types of directed energy.

5. The apparatus of claim 1, wherein the multiple energy emitting portals comprise a matrix and at least some of the matrix of energy emitting portals may be activated by the controller to emit directed energy independent of other energy emitting portals.

6. The apparatus of claim 5, wherein multiple energy emitting portals comprising the matrix are identifiable via predetermined coordinates selected from one or more coordinate types including Cartesian, Polar, number line, cylindrical, spherical, homogenous, curvilinear, orthogonal, skew log-polar, Plucker, canonical, parallel, barycentric, trilinear, and any transformation thereof, and wherein the controller may activate one or more of the energy emitting portals according to a pattern that may be associated with multiple predetermined coordinates.

7. The apparatus of claim 6, wherein the controller activates the source of directed energy to temperospatially deliver directed energy via emitting portals positioned proximate to at least one of a first portion of a region of interest and a second portion of the region of interest, and wherein the biometric measurement device provides feedback indicative of at least one of stimulation of and reduced activity in one or both of the first and second portion of the region of interest and adjusts at least one of the selection of energy emitting portals, the type of directed energy, the duration of the directed energy, and the level of the directed energy based on the feedback.

8. The apparatus of claim 1, wherein the directed energy emission device forms part of at least one of a multi-modal wearable cranial appliance, a wearable orthopedic appliance, a sleep-related appliance, an article of clothing, a patient imaging system, a hand-held device, a portable controller for administration and monitoring by a remote practitioner, and a wireless controller system.

9. The apparatus of claim 1, wherein the biometric measurement device comprises an electronic gaming device requiring inputs based upon cognitive analysis and manipulation of a user input mechanism.

10. The apparatus of claim 1, wherein the biometric measurement device comprises at least one of a electroencephalography (EEG) apparatus, an electromyography (EMG) apparatus, a thermometer, an electrodermography (EDG) apparatus, a photoplethysmography (PPG) apparatus, an electrocardiogram (ECG) apparatus, a pneumography apparatus, a capnography apparatus, a rheonocephalography (REG) apparatus, and a hemoencephalography (HEG) apparatus.

11. The apparatus of claim 1, wherein said predetermined directed energy therapy is capable of regulating the production of adenosine triphosphate (ATP) and is selected to at least one of up regulate and down regulate the production of ATP of cells within the target region of the mammalian subject.

12. The apparatus of claim 11, wherein the predetermined directed energy to up regulate ATP production is selected having a peak wavelength of at least one of approximately 620 nanometers, approximately 680 nanometers, approximately 760 nanometers, and approximately 820 nanometers.

13. The apparatus of claim 11, wherein the predetermined directed energy to down regulate ATP production is selected having a peak wavelength of at least one of approximately 750 nanometers, approximately 870 nanometers, approximately 900 nanometers, and approximately 950 nanometers.

14. The apparatus of claim 1, wherein said predetermined directed energy therapy is capable of regulating at least one of the efficacy and potency of at least one of pharmaceuticals, over-the-counter supplements, and medications to be administered to or by the mammalian subject and is selected to at least one of up regulate and down regulate at least one of the efficacy and potency of at least one of pharmaceuticals, over-the-counter supplements, and medications to be administered to or by the mammalian subject.

15. The apparatus of claim 1, wherein said predetermined directed energy therapy is capable of regulating at least one of endogenous stem cells, natural DNA sequences, synthetic DNA sequences, and associated genes, sub-sets and/or pre-cursors of said natural or synthetic DNA sequences and is selected to at least one of up regulate and down regulate at least one of endogenous stem cells, natural DNA sequences, synthetic DNA sequences, and associated genes, sub-sets and/or pre-cursors of said natural or synthetic DNA sequences.

16. The apparatus of claim 1, wherein said predetermined directed energy therapy is at least one of selected: (a) from a pre-existing list of potential directed energy therapies based on a pre-therapy assessment of said mammalian subject with reference to an aggregated database incorporating at least one of pre-therapy assessments of other mammalian subjects, directed energy therapies of other mammalian subjects, and results of the directed energy therapies of other mammalian subjects; and (b) by a user of said apparatus based on a pre-therapy assessment of said mammalian subject at least one of with and without reference to said aggregated database.

17. The apparatus of claim 16, wherein said pre-therapy assessment includes at least one of positron emission tomography (PET), computed tomography (CT), single-photo emission computed tomography (SPECT), blood biomarker testing, and cognitive testing to establish a functional baseline for the mammalian subject from which to compare the results of the predetermined directed energy therapy as determined by the biometric measurement device at least one of during and after the administration of the predetermined directed energy therapy.

18. The apparatus of claim 1, wherein the effectiveness of said directed energy therapy may be augmented by at least one of physical exercise and mental exercise at least one of before, during and after said directed energy therapy.

19. The apparatus of claim 1, wherein the directed energy emission device, the source of directed energy, the controller and the biometric measurement device of claim 1 are all part of a clinical directed energy system located in a first location, and further including a remote directed energy system located at a second location remote from the first location and including a second directed energy emission device, a second source of directed energy, a second controller, and a second biometric measurement device to collectively deliver the predetermined directed energy therapy as originated by the controller of the clinical directed energy system to the mammalian subject using the remote directed energy system.

20. The apparatus of claim 19, wherein said predetermined directed energy therapy is capable of regulating at least one of the efficacy and potency of at least one of pharmaceuticals, over-the-counter supplements, and medications to be administered to or by the mammalian subject and is selected to at least one of up and down regulate at least one of the efficacy and potency of at least one of pharmaceuticals, over-the-counter supplements, and medications to be administered to or by the mammalian subject, and wherein the clinical directed energy system is capable of: (a) accessing electronic health records of the mammalian subject before the application of the predetermined directed energy therapy; (b) calculating the characteristics of the energy of the predetermined directed energy therapy to be administered to the mammalian subject, wherein the characteristics of the energy include at least one of fluence, flux density, wavelength, waveform, pulse rate, and duration; (c) calculating a degree of modification to the amount of pharmaceutical, over-the-counter supplement and/or medication to be administered to the mammalian subject based on the increase or decrease in efficacy or potency due to the up or down regulation as a result of the predetermined directed energy therapy; and (d) communicating the degree of modification to at least one of a prescribing healthcare professional, a pharmacy associated with the mammalian subject, and the remote directed energy system.
